(19) United States
(12) Patent Application Publication
(10) Pub. No.: US 2017/0246472 A1
(43) Pub. Date: Aug. 31, 2017

(54) SYSTEMS, DEVICES, AND METHODS FOR TISSUE THERAPY

(71) Applicants: James C. CHEN, Portland, OR (US); Llew KELTNER, Portland, OR (US); Alexei N. NAIMUSHIN, Portland, OR (US)

(72) Inventors: James C. CHEN, Portland, OR (US); Llew KELTNER, Portland, OR (US); Alexei N. NAIMUSHIN, Portland, OR (US)

(21) Appl. No.: 15/508,844

(22) PCT Filed: Sep. 8, 2015
(86) PCT No.: PCT/US15/49030

§ 371 Date: Mar. 3, 2017

Related U.S. Application Data
(60) Provisional application No. 62/047,065, filed on Sep. 8, 2014.

(51) Int. Cl. A61N 5/06; A61M 37/00

(52) U.S. Cl. CPC A61N 5/062; A61N 5/0601; A61M 37/0092; A61N 2005/0626; A61N 2005/0612; A61N 2005/0602; A61N 2005/063; A61N 2005/067

(57) ABSTRACT

Systems, devices, and methods are described for providing, among other things, improvement in treatment of abnormal tissues including cancer, utilizing light activated drug therapy. Improvements include the use of optical penetration and drug penetration enhancers, in combination with photosensitizing drugs, and the use of localization aids and implements, which enable treatment of larger tissue volumes using minimal tissue access. The cytotoxic photoactivation process is also improved by using internal guidance to direct the light source to and at the lesion site using a naturally occurring orifice or intravascular access route.

### CROSS-REFERENCE TO RELATED APPLICATIONS

This application claims the benefit of U.S. Provisional Application No. 62/047,065, filed on Sep. 8, 2014, which is incorporated by reference herein in its entirety.

### SUMMARY

In an aspect, the present disclosure is directed to, among other things a medical device configured to deliver photodynamic therapy, activate a photosensitizing compound, deliver a photosensitizer, deliver an optical clearing agent, deliver a tissue penetration agent, and the like. In an embodiment, the medical device includes a needle having body structure having at least one internal surface defining a passageway. In an embodiment, the medical device includes a navigation assembly including circuitry configured to orient a distal end of needle within a target region. In an embodiment, the medical device includes a photo-activation assembly receivable within the passageway. In an embodiment, the photo-activation assembly includes at least one optical emitter. In an embodiment, the photo-activation assembly includes circuitry configured to generate one or more parameters for incrementally increasing a light dose. In an embodiment, the photo-activation assembly includes circuitry configured to generate one or more parameters for delivering a light dose sufficient to induce programmed cell death, without substantially inducing necrosis, of cells within one or more target regions proximate an outer surface of the needle.

In an embodiment, the medical device includes an acoustic assembly receivable within the passageway. In an embodiment, the acoustic assembly includes at least one transducer operable to deliver an acoustic stimulus to one or more target regions proximate an outer surface of the needle.

In an embodiment, the medical device includes a clearing agent assembly receivable within the passageway. In an embodiment, the clearing agent assembly includes at least one reservoir having an optical clearing agent composition. In an embodiment, the clearing agent assembly is configured to deliver the optical clearing agent composition from the at least one reservoir to one or more target regions proximate an outer surface of the needle. In an embodiment, the optical clearing agent composition includes one or more materials that reduces light scattering of tissue. In an embodiment, the optical clearing agent composition includes one or more of butanediols, dextrose, dimethyl sulfoxide, fructose, glucose, glycols, hypaque, mannitol, trazograph, and verografin.

In an embodiment, the medical device includes a penetration enhancer assembly receivable within the passageway. In an embodiment, the penetration enhancer assembly includes at least one reservoir having a tissue penetration enhancer composition. In an embodiment, the penetration enhancer assembly is configured to deliver the tissue penetration enhancer composition from the at least one reservoir to one or more target regions proximate an outer surface of the needle. In an embodiment, the tissue penetration enhancer composition includes one or more of DMSO and ethanol.

In an embodiment, the medical device includes a photosensitizer assembly receivable within the passageway. In an embodiment, the photosensitizer assembly includes at least one reservoir having a photosensitizer composition. In an embodiment, the photosensitizer assembly is configured to deliver the photosensitizer composition from the at least one reservoir to one or more target regions proximate an outer surface of the needle. In an embodiment, the photosensitizer composition includes one or more of methylene blue, porphyins, chlorins, phthalocyanines, tetrapyrroles, psoralens, flavins, tetracyclines, sulfa compounds, and photoactive prodrugs.

In an aspect, the present disclosure is directed to, among other things, a method including generating at one or more parameter for controlling at least one of a direction, intensity, duration, or peak emission wavelength of an illumination source responsive to acoustic information indicative of a tumor location, a tumor dimension, or tumor type. In an embodiment, the method includes interrogating one or more regions within a biological subject with an optical stimulus. In an embodiment, the optical stimulus is of a character and for a duration sufficient to activate one or more photosensitizers within one or more regions within a biological subject. In an embodiment, interrogating the one or more regions within a biological subject with the optical stimulus includes incrementally increasing a light dose. In an embodiment, interrogating the one or more regions within a biological subject with the optical stimulus includes interrogating the one or more regions with an optical stimulus having a dose sufficient to induce programmed cell death, without substantially inducing necrosis, of cells within the one or more regions within a biological subject.

In an embodiment, the method includes delivering a photodynamic therapy composition including at least one photosensitizer, at least one optical clearing agent, and a pharmaceutical acceptable carrier. In an embodiment, the method includes delivering a photodynamic therapy composition including at least one photosensitizer, at least one tissue penetration enhancer, and a pharmaceutical acceptable carrier. In an embodiment, the method includes delivering a photodynamic therapy composition including at least one photosensitizer, at least one tissue penetration enhancer, at least one optical clearing agent, and a pharmaceutical acceptable carrier.

In an aspect, the present disclosure is directed to, among other things, a photodynamic therapy composition including at least one photosensitizer and a pharmaceutical acceptable carrier. In an embodiment, the at least one photosensitizer includes one or more of methylene blue, porphyins, chlorins, phthalocyanines, tetrapyrroles, psoralens, flavins, tetracyclines, sulfa compounds, and photoactive prodrugs. In an embodiment, the photodynamic therapy composition includes at least one optical clearing agent and a pharmaceutical acceptable carrier. In an embodiment, the at least one tissue penetration enhancer includes one or more of DMSO and ethanol. Other non-limiting examples of tissue penetration enhances include short and long chain alcohols, amides, esters, fatty acids, fatty acid esters, liposomes, which may optionally contain photosensitizers, phospholipids, pyrrolidones, surfactants, urea and derivatives, cyclodextrins, oxazolidinones, monoolein, vitamin E, terpenes, iminosulfuranes, and the like. In an embodiment, the at least one optical clearing agent includes one or more of butanediols, dextrose, dimethyl sulfoxide, fructose, glucose, glycols, hypaque, mannitol, trazograph, and verografin. In an embodiment, the photodynamic therapy composition includes at least one tissue penetration enhancer and a pharmaceutical acceptable carrier.

In an aspect, the present disclosure is directed to, among other things, a photodynamic therapy device including an optical clearing agent delivery component. In an embodiment, the photodynamic therapy device includes a tissue penetration enhancer delivery component. In an embodiment, the photodynamic therapy device includes a photosensitizer delivery component. In an embodiment, the photodynamic therapy device includes an acoustic stimulus delivery component. In an embodiment, the photodynamic therapy device includes an optical stimulus delivery component.

### BRIEF DESCRIPTION OF THE FIGURES

FIGS. 1A-1J are perspectives views of medical devices according to one or more embodiments.

FIGS. 2A-2C are perspectives views of medical devices according to one or more embodiments.

FIGS. 3A-3D are perspectives views of medical devices according to one or more embodiments.

FIGS. 4A and 4B are perspectives views of medical devices according to one or more embodiments.

FIGS. 5A-5D are perspectives views of medical devices according to one or more embodiments.

FIGS. 6A-6D are perspectives views of medical devices according to one or more embodiments.

FIGS. 7A and 7B are perspectives views of medical devices according to one or more embodiments.

FIG. 8A and 8B are perspectives views of medical devices according to one or more embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Photodynamic therapy (PDT), light activated drug therapy (LADT), and the like describe technologies and methodologies that utilize the combined action of electromagnetic energy and at least one active agent to effect a therapeutic action, and is used most often against human and animal cancers. In an embodiment, during operation, coherent or noncoherent energy sources such as a laser, a light emitting diode, and the like generate electromagnetic energy to one or more regions and activate photosensitizing compound or molecule capable of absorbing the light. In an embodiment, interaction of drug molecules and light photons in the presence of molecular oxygen leads to generation of singlet oxygen and other radical species, which are cytotoxic. The cytotoxic action occurs when reactive species are generated in sufficient quantity to irreversibly and overwhelmingly affect cell function and integrity. Molecular oxygen is important and pivotal for the PDT/LADT effect, and can be consumed rapidly when using light at high fluence rates. Rapid consumption of oxygen reduces the effectiveness of therapy, even when light and drug present and continuing to interact. Lower fluence rates are sometimes employed for treatment of larger cancerous lesions, which necessitate a prolonged light delivery period, in order to deliver an adequate total light dose to achieve a therapeutic effect. The prolonged light delivery period necessary to enhance the therapeutic effect of low fluence rates poses major practical clinical obstacles and concerns, and safety as well as convenience issues. To date, no adequate and practical solutions have been proposed to solve the prolonged fluence conundrum, other than what is detailed in the present disclosure.

In an embodiment, to effectively treat large, bulky tumors, whether they are benign or malignant in character, the total light dose must be above a threshold that results in cell death. In general, the larger the lesion, the greater the total light dose required for treatment. Traditionally, PDT/LADT has been administered to large tumors by using high fluence laser light sources in order to attempt to deliver an adequate total light dose. The greater the light dose required, the greater the fluence rate must be, in order to keep the treatment time within a clinically practical, lower risk range. But at least two major problems result from this dictum. First, tissue and blood oxygen is consumed too rapidly leading to premature cessation of the photoactivation process, which results in incomplete treatment and survival of the abnormal cells and tissue as mentioned. Secondly, the high fluence rate produces a necrotic effect due to overwhelming damage to the cells, which do undergo cell death, especially affecting the cells proximate to the light source. These cells receive a very large light dose which leads to an excessive amount of singlet oxygen generation, leading to necrosis. In contradistinction, the cells more distant to the light source may well survive due to the oxygen consumption issue. Necrosis is an undesirable consequence of the high fluence rate, since this process also results in inflammation, which has been shown in multiple studies to be a secondary tumor promoting process. In an embodiment, necrosis and inflammation is avoided by proper dosing of drug and light. Accordingly, in an embodiment, the present disclosure details how to avoid overtreatment of the lesion close to the light source, and under treatment of tissue further away, or distant to the light source. The paradox of high dose, rapid light delivery resulting in both over and under treatment, and other issues and problems mentioned in this disclosure has resulted in the view of PDT/LADT as a therapy with very limited clinical application, and with virtually no practical way to apply it to the treatment of more advanced cancer deposits, characterized by larger, bulky lesions.

There is a need for closely spaced multiple light sources in order to treat higher volume lesions. Limited depth of penetration of light is a major obstacle when using PDT/LADT for bulky, high volume lesions. The methodology of utilizing multiple closely spaced light sources also poses significant inconvenience, procedural risk, increases patient discomfort and pain, and is not at all practical or feasible in typical cancer treatment settings. The technical expertise and equipment needed to accomplish PDT/LADT using this methodology is lacking in the vast majority of oncology treatment settings. Prolonged periods of imaging required for multiple device insertion frequently include the use of techniques involving imaging contrast agents and ionizing radiation, which increases the risk of adverse drug reactions, organ failure, and secondary cancers. Intensive use of image guided device placement also entails prolonged periods of enforced patient immobility, which is very problematic in the settings of patients with advanced cancer. Imaging facilities such as fixed CT scanners, MRI units and the like are tied up for long periods of time for a single patient, which is impractical in the typical clinical setting. Even when the expertise and hardware are available for this mode of PDT/LADT, the precision required is not always achieved, and undertreatment and overtreatment results are the norm. Prior to the present disclosure, treatment of large lesions required multiple devices, each associated with a very limited effect zone, prolonged use of imaging equipment, and overall increased risk, discomfort, and clinical impracticality, previously taught to treat localized bulky cancers.

Another major hurdle confronting the use of PDT/LADT is that treatment relies on systemic drug delivery, which has the downside of nonspecific and generalized tissue photosensitization. This can lead to unwanted normal tissue photoactivation in organs such as the skin surface, which may lead to pain, scarring, and other undesirable tissue injury effects. Instances of cutaneous injury have occurred, and if light is delivered to an internal lesion such that the zone of effect is extended to normal tissue external to the lesion, then unwanted normal tissue damage adjacent to the treatment zone could and has occurred, in some cases leading to serious morbidity and even death. In addition, the number and timing of treatments is very limited when using systemic drug dosing, due to the need to allow a significant amount of time to pass for complete drug clearance from the body. Otherwise, the patient could be treated with an excessive and dangerous amount of photosensitizing drug due to excessive systemic drug accumulation. If a medical condition arises such that the treatment must be delayed, after systemic drug injection, a window of treatment opportunity is lost and the patient is exposed to generalized photosensitization risk, especially of the skin, for no therapeutic gain. If the patient requires an unexpected or emergency procedure after systemic photosensitization, this situation can be very dangerous and problematic, since virtually all medical procedures and operations require some form of illumination in order to carry out the intervention. The illumination for the intervention can result in inadvertent normal tissue photoactivation damage, and the need for tissue shielding from light may render the intervention more risky, and more difficult to perform, potentially resulting in a poorer outcome.

The present disclosure details one or more methodologies or technologies for attenuating nonspecific photosensitization and photoactivation leading to a very specific and circumscribed treatment, which can be performed at any convenient time, in a virtually unlimited fashion, without incurring further risk in the event of an emergency or unplanned urgent intervention. In an embodiment, systemic drug of photodynamic drugs has been used to ensure delivery of adequate drug throughout the lesion to be treated. In an embodiment, localized drug delivery has been used for thin, superficial disease, or for treatment of very small lesions. Experiments have shown that localized injection of photosensitizing drug in large lesions leads to uneven and unpredictable drug distribution. In an embodiment, the present disclosure describes drug formulations that enable localized, more uniform, and clinically efficacious drug dispersion in large, bulky lesions.

As mentioned previously, conventional interstitial PDT used to treat a bulky lesion relies on the precise placement of multiple closely spaced light sources, most commonly optical fibers in a predetermined array. The use of multiple light sources adds treatment complexity, discomfort, and risk to the procedure, such as bleeding, infection, unwanted and dangerous organ perforation, and increased chance of device dislodgement, since multiple punctures with multiple devices to reach and treat the site are necessary. The use of multiple light sources can lead to inadvertent unwanted tissue damage due to the multiplicative effect of overlapping light fields. Dislodgement of devices can lead to dangerous normal tissue photoactivation damage, and undertreatment of the target site, since less light is received in the tissue to be treated than originally planned for. Placement of multiple light sources also increases the chance of tumor seeding along the multiple placement tracks, a well-known medical risk from punctures involving entry into tumor parenchyma. The present disclosure details novel embodiments, along with new methods, which can be used to treat bulky, high volume lesions using a single entry point, which reduces the risk of harm, easier, more efficacious, and more comfortable for the patient and which afford significant clinical advantages.

An additional unresolved issue with light therapy is that light emitted by a single light source is limited in depth and volume due to inherent tissue optics characterized by enhanced light absorption and scattering in biological solid tissues. Accordingly, some have used multiple light sources in order to achieve a significant treatment zone, with attendant risks and downsides involved with multiple tissue punctures.

In an embodiment, one or more methodologies or technologies include enhancing light penetration and delivery within tissue in a novel manner, which greatly increase and enhance the zone of effect enabled by a single device used to treat a localized lesion. In an embodiment, one or more methodologies or technologies not only significantly and meaningfully increase the total volume that can be treated, but also details how to maximize the efficacy of therapeutic effect within the expanded treatment zone.

When attempting to treat large tissue volumes, defined as greater than 2 cm diameter lesions, various studies have demonstrated that illumination time can be very prolonged, due to the need to use a relatively low, as opposed to a high fluence rate. Research has shown that using high fluence rates and short illumination times in order to increase clinical practicality and practicability leads to rapid oxygen consumption and subsequent highly undesirable rapid recanalization of blood vessels closed by the light activated drug therapy. Recanalization of blood vessels, which reestablishes blood flow can lead to increased tumor cell survival, thus obviating the desired treatment effect. Thus, it has been ascertained in multiple studies that more prolonged illumination times are required in order to increase treatment efficacy, along with the use of multiple closely spaced light emitting devices. The present disclosure details technologies and methodologies to reduce the illumination time significantly, to a range that is more clinically practical, while enhancing and maintaining efficacy of treatment.

Another critical issue relating to medical devices used internally is that they must be safely and accurately delivered to the treatment site, and successfully retained at the treatment site for the duration of therapy. If devices are inaccurately placed, or move or shift location during treatment, serious side effects and even mortality could occur. In an embodiment, the present disclosure include a means of tracking the device during delivery to the target location, monitoring proper retention at the treatment site, and provides a means of securing the device during treatment. In an embodiment, routes of entry into the body includes percutaneous, through an orifice, via intravascular channels, and the like. Another neglected aspect of treatment planning and PDT/LADT device insertion and placement relates to the use of tissue phantoms and virtual imaging, which is also described in this disclosure.

In the case of large cancerous deposits, prior studies and clinical practice teach that the entire lesion must be effectively treated with light and drug. This teaching leads to extremely complex light and drug dosimetry calculations and specialized equipment and technical know-how requirements that are not feasible in the vast majority of cancer treatment facilities. The requirement to treat the entire lesion also increases the risk that the treatment zone will exceed the lesion and result in unwanted damage to normal tissue external to the target treatment site.

An added unaddressed issue with PDT/LADT when applied to large lesions is that the effect zone is unpredictable. This factor precludes its use in many situations where lesions are treated adjacent to critical anatomic structures. Standard medical practice of treatment of large benign and malignant masses encompasses an element of predictability, which is important for treatment planning The lack of predictability of PDT/LADT is addressed in this disclosure, using tissue phantoms and treatment simulations to improve predictability and utility against larger masses. The present disclosure details relatively simple to use techniques that improve on past therapy, which is normally based on the use of highly complex, expensive, and difficult use of specialized equipment and the requirement for technical know-how and expertise how rarely available in the vast majority of medical treatment locations.

Prior PDT usage teaches the use of straight fiberoptic/laser systems for interstitial tumor treatment. This is a serious limitation, which restricts the access and entry points on the body, and risks injury to critical anatomic structures as the device is being passed in a straight line. The present disclosure teaches away from this problem and provides a new solution, which entails the use of curved and flexible guidewires and devices, which allow for more flexibility of access and avoidance of critical structures.

Accessing large, deep, internal mass lesions percutaneously can be very problematic in the clinical setting. A less risky, painless access route is not available in some patients, and awkward angles and points of entry may necessitate long needle passages with increased risk of bleeding, perforation of critical structures, increased pain, prolonged imaging and procedural time, and increased requirements for the patient to remain motionless in an abnormal position or posture which can render the treatment much more difficult, or not feasible at all. Furthermore, device dislodgment, migration, and dislocation can occur when percutaneous placement is utilized, and the device can migrate in the subcutaneous tissue compartment or deeper, without being apparent on the skin surface. Unwanted device motion can be very dangerous, leading to damage to normal tissues and normal organs from the device itself, or from nontarget photoactivation. The present disclosure details device embodiments and methods, which provide an elegant solution, by utilizing intravascular access to the mass lesion, which can be followed by extravascular access if required, for example if a tumor feeding vessel is not available, once the device is in lesion proximity. Fixation of the device is simplified and secure using the intravascular route, and device migration is mitigated simultaneously, since it is far more difficult for the device to back up once secured within a blood vessel conduit.

An added feature that is taught by this disclosure is the ability to use three dimensional printing techniques and equipment to manufacture customized devices at the point of care, or on site and on demand. The ability to perform this unique function provides clinical and economic advantages, and convenience not previously described for these types of light and drug delivery devices. The equipment and know-how is increasingly available, and suitable polymers can be employed to create biocompatible and custom sized and dimensioned devices that are suited to each patient's unique anatomic and physiologic requirements. All device components can be created on site as needed, which is very novel for this type of therapy.

The present disclosure details embodiments, technologies, and methods of PDT/LADT, which are utilized to treat large lesions. In an embodiment, technologies and methodologies include practical, efficacious, and convenient therapy of mass lesions currently untreatable with current therapies.

### Localized Drug Delivery Device Embodiments

In an embodiment, the device is designed in such a manner as to enable specific and targeted drug delivery of the photosensitizer, in a prespecified concentration, to a prespecified tissue volume. Common use of PDT and LADT employs systemic photosensitizer drug (PD) administration, almost always delivered by the intravenous route, which does not enable a uniform and controlled drug concentration to be achieved due to variable pharmacodynamic and pharmacokinetic effects stemming from patient physiology differences. Localized PD delivery can also specifically target a certain predetermined tissue volume to be treated, which is also not possible when using systemic drug delivery. Systemic PD delivery has the further disadvantage of unwanted systemic or localized side effects independent of photoactivation in nontarget tissues.

Though the localized delivery of PD has been described and can be adequate for very low volume lesions, the hurdles of controlling the depth and concentration of drug delivery, and increasing the depth of drug penetration from a single source for larger lesions have not been addressed. Prior studies have documented the heterogeneity of PD drug localization even in very low volume lesions when utilizing localized delivery with a conventional needle, where PD is delivered only from the distal end. Even greater heterogeneity of drug distribution would be expected in larger lesions, which has been observed by the inventors in large volume tissues injected with PD using a conventional needle.

### Other Device Embodiments

In an embodiment, a treatment device is conformed into a needle or sheath shape, and can be composed of suitable a materials including, among others, stainless steel, nitinol, biocompatible polymers of all types, and the like. In an embodiment, a needle will contain side holes, which are of variable shape, configuration, and diameter, as well as variable in number and configuration/pattern. The side holes permit egress of the drug into specific tissue volume, achieving the desired tissue PD concentration. Along the surface of the needle, the side holes can be augmented by dimples or nipple-like surface projections, which will reduce tissue clogging of the side holes, and increase the distance that the drug can be delivered, due to the nozzle like shape of the projections. By varying the speed and force of injection, the drug penetration depth into surrounding tissue is controlled.

A series of controlled experiments in vitro and in vivo are conducted in order to determine the optimal shape and configuration of side holes and PD delivery that enable the desired pattern of PD distribution. The side holes can be in the shape of tiny blunt nozzles that aid injection by increasing delivery pressure. The injection of PD may be manual or automated using automated injection devices such as syringes and other types of reservoirs, located on the proximal end of the needle device.

The distal tip of the needle can be nozzle shaped with a constricted tip, in order to reduce the chance of tissue plugging the tip as the needle is advanced. The side holes can be nozzle shaped in order to increase the pressure at which the drug fluid is ejected, thus increasing the effective drug delivery range. Use of needles to internally deliver drug allows for differential drug distribution in tumor or masses to be treated in a manner not possible with systemic drug delivery.

In an embodiment, the needle is rigid and in another embodiment the needle is flexible. In either case, the needle can be positioned at one boundary of the lesion, and then moved towards a different boundary, while drug is injected, thus insuring a more even and complete drug distribution within the lesion, from a single entry point into the lesion. Furthermore, the flexible needle can be withdrawn or advanced in different trajectories off of the original device axis, which also enhances the volume of tissue drug distribution. These embodiments and methods obviate the requirement of multiple lesion puncture sites, which reduces risks including bleeding and organ perforation, and reduces procedural time needed for multiple device placements, which also can reduce the anesthetic requirement.

Another embodiment employs a guidewire, which can be either metallic or polymeric. The guidewire fits into the interior of the drug delivery device starting at its hub, also known as an “over the wire” design, with the drug delivery device comprising essentially a sheath with side holes, whereby the guidewire allows for ease of redirection of the sheath path. Alternatively, sheath is directed by the guidewire at its tip, as in a long rail or monorail system, where the guidewire enter the sheath via a side hole located distally. This system enables the so-called rapid exchange, which is well established in the art.

A guidewire can be introduced into the intravascular compartment via a standard route using the femoral or radial artery as an example. Once introduced, the guidewire can be used to access a tumor feeding vessel. If it is deemed desirable for the device to be placed within the lesion substance, a sheath can be advanced over the guidewire using a variety of techniques and methodologies. At this point, drug can be administered intra-arterially and light delivered in a forward facing fashion. Tissue scattering aids in light beam dispersion, and the forward launching light source can be slightly angled and rotated in order to widen the beam diameter, which serves to increase the abnormal tissue volume treatment zone. Alternatively, the guidewire can be used to penetrate the abnormal tissue to be treated, by exiting the intravascular space and entering the tissue to be treated interstitially. This is facilitated by also introducing a sheath with an elongated, stiffening balloon located at the distal tip. The balloon obviates bleeding by blocking the arterial flow, and provides a stiffer channel for the guidewire to be inserted through, into the tissue, from the intravascular compartment into the extravascular region. Then, leaving the balloon inflated in order to prevent bleeding, a second sheath can be introduced over the guidewire and advanced into the substance of the lesion. This maneuver allows for interstitial drug delivery, introduction of an optical fiber or alternative light source, and lesion treatment.

In an embodiment, the PD can be mixed with a water soluble substance, or diluent prior to injection, such as mannitol, or glycerol, which have been shown to increase the optical penetration depth, when injected into tissue. For example, a 1% mixture of methylene blue can be combined with an aqueous mannitol or glycerol solution, which is part of the diluent for the methylene blue. Other optical penetration enhancers that also enhance tissue penetration of drug such as dimethyl sulfoxide (DMSO) can be used. Non-limiting examples of photoactive agents and classes of photoactive agents include photosensitizers, methylene blue, porphyins, chlorins, phthalocyanines, tetrapyrroles, psoralens, flavins, tetracyclines, sulfa compounds, photoactive prodrugs, and the like, and derivatives thereof.

Non-limiting examples of optical clearing agents include materials that reduces the scattering of tissue and make tissue more transparent, translucent, or the like. In an embodiment, scattering of tissue is reduced by diffusing of optical clearing agents with higher refractive indices and higher osmolality into tissues will match the refractive indices of tissue components with extracellular fluid

Further non-limiting examples of optical clearing agents include glucose, dextrose, fructose, glycols, butanediols, and x-ray contrast agents such as verografin, trazograph, hypaque and other imaging enhancers. The optimal concentration of optical clearing agent can be determined experimentally, in vitro and in vivo. In an embodiment, the combination of a photosensitizer and an optical clearing agent also enhances the ability to detect the drug location and degree of dispersion in tissue using standard imaging, compared to photosensitizer alone. Drug penetration enhancers such as dimethyl sulfoxide (DMSO) or ethanol along with optical clearing agents serve to enhance the volume of treated tissue, and increase the efficacy of therapy in a very novel way, not taught by prior use regarding treatment of large, internal tumors and masses.

Further, the device may optionally incorporate or accommodate an optical fiber or light distributor within the hollow needle shaft or sheath. In one embodiment, the distal end of the fiber is shaped to occlude the distal end of the injection needle device, which serves to control and regulate the flow of PD into tissue by enabling drug delivery only through the side holes. Without the occluding end, drug could be uncontrollably and unpredictably delivered preferentially out of the distal end of the needle. PD can be injected around the shaft of the optical fiber or light source, which includes non-limiting examples such as lasers, laser diodes, laser fibers, light emitting diodes (LED), polymeric light sources, incandescent, halogen, and other light emitting sources. The fiber serves to deliver the light energy needed to activate the PD, after the drug is delivered and distributed to the desired treatment tissue volume. The light is generated proximally as in the example of a laser diode coupled to an optical fiber, or distally, as in the example of an LED array located at the device tip.

A polymeric optical fiber can also be fashioned with an internal embedded nitinol guidewire within the center of the fiber axis. In this embodiment the optical fiber with a forward light launching capability or a cylindrical diffuser is placed simultaneously as the guidewire is positioned at the treatment site.

The method of needle placement may utilize one method of either, freehand insertion, laparoscopic guidance, robotic guidance, electromagnetic navigation, or standard image guidance. After drug injection, the light source can be positioned within the lesion, or on the boundary of the lesion. The light source can be moved along the drug injection track in order to increase the volume of light distribution without the need for multiple puncture sites.

In order to accommodate the internal light fiber and enable drug injection, a side vent is created on the device surface in its proximal end, enabling drug to be delivered within the needle shaft, around the fiber whose outer diameter is smaller than the inner diameter of the needle. This enables drug to flow around the fiber and out through the side holes. Optionally, the fiber can be inserted via the same injection port or a different access port on the device after PD injection.

The optical fiber may be composed of any of the known optically useful substances from glasses to polymers, and may be used to convey light of any wavelengths to the treatment site. When shorter wavelengths are used, such as in the blue waveband which has a very short tissue penetration depth, tissue penetration may be increased in a novel way due to the prior PD injection with the optical clearing agent. The drug embodiment in this case acts both as a PD and to increase the optical penetration depth in a very novel way. In alternative embodiments, the optical fiber can be positioned within the needle, or in another configuration, within a separate channel within the needle internally parallel to its long axis.

All components can be engineered and constructed to different lengths and diameters to whatever the desired specifications, and can be stiff or flexible. The needle/optical fiber can be delivered to the treatment site percutaneously, through an orifice, by way of an endoscope, robotically controlled, or through the vascular system. The assembly can optionally incorporate a distal steering mechanism, which is adjustable as the device is advanced. Another embodiment utilizes a robotically controlled delivery system.

In another embodiment, the light emitting device is composed of a shape memory material, such as nitinol. The device is capable of being delivered percutaneously via a needle or a trocar. As the device exits the needle it takes a shape of a spiral. A spiral can either be a constant diameter spiral (forming a cylinder) or have a variable diameter of its turns. For example, it can form a shape, which resembles a football, with the larger diameter in the middle, and smaller diameter towards the ends. Having a variable shape device would allow in treatment of lesions, which are of spherical or irregular shape. Such shape memory device could be combined with other device features described elsewhere in this disclosure. Various drug delivery methods could be practiced with this device.

### More Drug Delivery Needle Embodiments

For drug delivery to larger tissue volumes, the needles with or without multiple side holes can be pre-curved, or steerable, or shaped in a coiled configuration.

The coiled configuration needle is preferably rigid, but optionally can be flexible. The needles can be delivered manually, or via a motorized or automated driver system. The coil can be of various diameters and pitches. The coil design enables treatment of a large tissue volume by encompassing a drug treated zone, which is outside of an inner zone, which does not contain drug. The light activation of the drug creates a shell of devascularized tissue, which encompasses the non-drugged tissue, thus effecting a much larger treatment zone with less drug, and without the need for light delivery to the interior of the lesion. This very efficient treatment configuration leads to less drug exposure and enables a significant tissue volume to be treated by a single pass of the drug delivery device, minimizing the number of punctures of tissue, compared to using a straight needle, which could require multiple passes to deliver drug to a large tissue volume.

When drug is delivered locally, the light source can be positioned anywhere outside of the tissue volume containing drug. As long as light can penetrate the intervening tissue, it can be used to photoactivate drug at a distance to the light source. Therefore, light can be delivered from within the drug loaded tissue, or at a distance to the drug loaded tissue. For example an optical fiber designed and configured to launch light in a forward direction is positioned at tumor edge, and light emitted through the light device tip is scattered in a forward direction. In another method, if the tip of the device is positioned near the more distal portion of the tumor, the device can be gradually withdrawn proximally, which essentially allows a much greater volume of tumor to be illuminated.

Drug can also be delivered, as the spiral device is being advanced in tissue or withdrawn in tissue. This process serves to disperse the drug over a period of time while the device is in use. A spiral device design obviates the requirement for multiple needle punctures, which reduces the risk of bleeding, infection, and discomfort.

An alternative device is in the configuration of a screw with variable width flanges. The flanges are able to convey drug to the outer edges of the device. The core of the screw can be hollow enabling drug to be conveyed to the flanges.

Tissue penetration can be optionally facilitated, by incorporating a means of heating the distal tip of the needle using an electrical means. Ultrasonic vibration can also be used at the distal needle tip. Both of these means can be used to cut through the tissue, which may be as hard as bone, in order to generate a needle track.

Another embodiment uses a pre-curved channel housed within the outer most device sheath. The sheath is angled or curved such that the drug delivery needle will follow an angle, curve, or track that is off of the main axis of the device. In this embodiment, a single puncture at the skin level, or entry point into target tissue through an orifice, or through a blood vessel can be used to direct the needle in different trajectories, thus enabling increased drug dispersion to a larger tissue volume. The same track can be used to change the location of the light delivery device, following the same path off axis as the drug delivery needle. This also enables increased light delivery to a greater volume of tissue through a single entry point on or within the body.

### Light Dose Ramping

Ramping up the light dose over a period of time can unexpectedly increase the efficacy of light activation.

The ramping effect serves to gradually increase the zone of effect by increasing the tissue volume which receives light over time, by reducing blood flow in an ever increasing radius. The photoactivation process which is proximate to the light source and serves to decrease or stop blood flow, and also to photobleach the photosensitizer, two factors which also can increase the effectiveness of light absorption more peripherally, since collapsed blood vessels in tissue allow more transmission of light and photosensitizer no longer absorbs light after photobleaching. This process will increase the effective light penetration distance due to reduced photon absorption closer to light source, which results from vascular stasis. This modulation of the photoactivation effect allows the effect zone to be monitored in real time if desired, for example using imaging such as ultrasound, which can detect changes in echogenicity and blood flow resulting from photoactivation. This method also allows for a shorter wavelength of light to unexpectedly penetrate tissue more effectively (shorter wavelengths of light penetrate tissue less well than longer wavelengths), and comparably to a longer wavelength of light. Ramping can be performed in a series of steps, with each step constituting an increased light intensity or fluence rate, or be a smoothly increased rate of light delivery. Control of the light dose increase can be accomplished manually, in an automated fashioned, or be preprogrammed.

Compared to a steady fluence rate, light dose ramping unexpectedly can result in a greater effect zone when the same total amount of light is delivered in both methods.

### Distal Retention Balloon

A balloon inflated at the distal portion of the device can also unexpectedly increase the zone of light penetration. This has been shown in an experiment that utilized different diameter light sources emitting an identical total amount of light. The device with the greater diameter unexpectedly produced a greater effect zone compared to the thinner diameter device, due to tissue compression. Balloons used during PDT/LADT are only effective against very superficial and low volume disease, such as the lining of the bladder, or within the lumen of a diseased blood vessel. The use of balloons in the present disclosure, reveal how the zone of effect is expanded to a significant degree, far beyond that which was previously taught. The use of balloons to treat very thin, intravascular lesions, with treatment purposely limited in scope to a thin layer adjacent to the balloon. The present disclosure teaches away from this, and in fact discloses the use of balloons to greatly expand the treatment zone. The balloon can be positioned circumferentially around a cylindrical diffuser or unclad portion of optical fiber, or at the distal tip of the device in case of forward directed light, which is aimed into the balloon scattering light in the direction the fiber tip is aimed. A variety of shapes, sizes, and balloon dimensions are contemplated. The balloon is reversibly inflated with air, or light transmissible fluid, which is preferably biocompatible. Fluid within the balloon also serves to cool the heat generated by the light source within the target tissue, preventing balloon damage or rupture.

Furthermore, the balloon when inflated prevents rapid reestablishment of blood perfusion and flow, which is a phenomenon and drawback seen with short light illumination times. It has been found experimentally that longer illumination periods can be more effective compared to shorter illumination time due to a variety of reasons including rapid reperfusion of the treatment site. This occurs due to blood clot and thrombus breakdown occurring from the physiologic anticlotting mechanism. However, short illumination times are a clinical goal, whose advantages include reduced chance of device dislodgments, increased patient comfort, reduced immobilization time, reduced risk of infection, and enhanced clinical utility and convenience. Thus, use of an inflated balloon has multiple novel actions, including reducing treatment time, expanding zone of effect, and reducing device dislodgment risk.

### Drug Delivery Methods

Drug can be delivered intermittently with each delivery bolus followed by photoactivation. The photoactivation process may facilitate subsequent drug delivery by enabling drug to penetrate further from the device due to breakdown of cells and cell cohesion, which in effect increases the interstitial space for drug egress. Thus, one or more cycles of drug delivery followed by photoactivation may serve to increase the zone of treatment effect in a controllable manner. The force of drug egress can also be varied by adjusting the force which ejects the drug, which is applied to the device lumen.

By ejecting and thus delivering drug simultaneously from multiple locations along the needle shaft, more complete distribution of drug within the lesion occurs. Even in the event of nonuniform drug distribution, a volume tissue containing no drug or a subtherapeutic amount of drug can be effectively treated. This event occurs, provided that other tissue external or distal to the non drug containing tissue does receive adequate drug and light, due to the drug and light distributor device design.

### Additional Device Configurations and Designs

In an embodiment, the device includes a straight needle having a tip that is beveled or cone shaped. In an embodiment, the device includes needle that is closed at its tip or optionally open at its tip. In an embodiment, the needle is flexible or rigid and composed of metal including, among others, stainless steel, or composed of a polymer. Optionally an optical fiber or other thin light source is contained within the needle. The light device can be inserted reversibly or irreversibly into the needle shaft, or the light device can be incorporated within the needle. In this case, the diameter of the light device is small enough to allow fluid containing drug to flow around it. Light can be delivered through the needle by way of the multiple side holes, which also serve to disperse the drug over a volume. Light can also penetrate the needle if the needle is optically transparent, for example if composed of a polymer.

### Ultrasound Transducer

The optional ultrasound transducer, which is incorporated into the device, or inserted into the device can have multiple functions. In addition to use for localization of the device within the treatment area, the ultrasound transducer can monitor the therapeutic effect using standard imaging/processing techniques such as color flow Doppler to assess blood flow or treatment induced flow stasis. The transducer can also be used to enhance drug penetration into tissue. The transducer can also function to enhance device penetration into tissue at the distal device tip by breaking up tissue at the tip or by heating tissue at the tip. So, in addition to imaging, the ultrasound transducer can generate cavitating gas bubbles and microbubbles that permeabilize tissue for drug delivery. The introduction of gas bubbles into tissue also enhances light penetration into tissue, and can provide an enhanced drug activation effect in addition to the light photoactivation effect. Single or multiple US transducers are incorporated into the sheath, or can be powered by, or on separate wires and circuits within the sheath.

Multiple US transducers can be incorporated into the device in order to increase the ability to assess the completeness of treatment. By using different spacing schemes of the US transducers, the treatment site is evaluated before, during, and after treatment. Color flow Doppler, or power Doppler can be used to evaluate blood vessel closure due to treatment. Other US changes due to treatment can be detected with increased sensitivity due to the immediate proximity of the transducer to the treatment site.

For enhancement of device placement, the ultrasound can be focused down to a very narrow, intense beam resulting in heating which aids in driving the device through tissue. The transducer can also be used to locate the device in transit to the target area, at the target area, and to confirm that the device has not dislodged from the target area. The beam can be directed externally for localization purposes.

Ultrasound can be used to detect the excursion and degree of penetration of photosensitizing drug into tissue. The use of multiple US transducers increases the ability to detect signal differences and changes induced by drug penetration, and the subsequent treatment effects in real-time. Thus, US can be used throughout the procedure, to localize the device, monitor drug distribution, and assess the treatment effect. Studies can be performed to correlate US signals and signal changes to the various stages and outcomes of the procedure.

Ultrasound imaging can be used to monitor and guide delivery of the photosensitizer drug, since the altered tissue characteristics of the drug within tissue can be distinguished from surrounding non drug containing tissue. Ultrasound imaging is also facilitated, by surrounding the transducer with fluid, within the device lumen. Non-limiting examples of transducer include Piezoelectric Ultra Sound (US) tranducers, Capacitive micromachined US transducers, and Photoacoustic US transducers.

The ultrasound device can be a part of the guidewire, or on a separate thin wire circuit, and is inserted and removed from within the sheath or catheter.

### Light Source and Optical Fiber

The coherent or noncoherent light source can be used to photoactivate the drug, and can be used for device placement, if infrared light, which penetrates tissue is used. The actual source of light is internal within the body, or external to the body. In an embodiment, polymeric or glass optical fibers can be utilized, along with hollow optical fibers, which can be placed using a guide wire within the fiber. Specially designed hollow optical fiber can also be used as the means of drug delivery, by utilizing the hollow core as the conduit for drug. The light source can be incorporated into the sheath or catheter, which is also used to deliver the drug, or can be a separate, for insertion and removal within the sheath or catheter.

### Localizing Coil

An electromagnetic coil, which is well known, can be incorporated as an external winding on the device, or encased within the outer sheath of the device. The detection coil, which can also be a system of multiple coils, are positioned externally on the body surface. They are utilized to track the position of the device and monitor the ultimate position of the device, to reduce the chance of unnoticed dislodgment. The signal strength sensed by each detector is used to determine internal device position. An algorithm can be developed based on the known detector signals strength, which varies with distance. If the signal strength correlation and relationship to distance is known by testing and tabulating in tissue phantoms, then the device position can be triangulated, with accuracy increasing with the number of detectors, which are arrayed in different body locations. Other intracorporeal navigation systems are well described and known as well. The current navigation and positioning system described has the advantage that the line of sight requirement is not necessary, as is described and utilized by optical navigation systems.

Being able to ascertain the location of the device in realtime using the electromagnetic system obviates the need to subject the patient to repeated MRI or CT scans to confirm the device position, as it is being placed, and to confirm correct positioning during light activation. Repeated imaging is inconvenient, costly, not practical in a busy clinical setting where other patients require imaging for diagnosis, exposes the patient to potentially dangerous ionizing radiation in the case of CT, and may require the repeated use of contrast agents, which also poses risks as well. From viewed from an external visual vantage point, device back out can occur with no visible positional change in the external portion of device, since the backup can occur deep to the skin. Though ultrasound can also be used to localize the device internally, there are situations where the image is degraded or not even feasible due to imaging through a gas filled or hollow organ such as the lung, bowel, or bladder. In a very large patient, ultrasound may also not allow for adequate image resolution to be clinically useful, due to beam attenuation over distance.

### Tissue Phantoms and Treatment Simulations

Simple tissue phantoms used in PDT/LADT have been described. A physical tissue phantom is utilized to assess injected drug concentration, and light distribution in order to optimized device design, in terms of drug dispersion and light delivery. In addition, preclinical in vivo testing can be utilized to determine the optimal parameters for drug injection, drug concentration and composition, light delivery and overall device design. For example, the following attributes and parameters are determined preclinically (in vitro and/or in vivo, using laboratory bench top testing, or in tissue models):
Composition and concentration of PD with or without optical clearing agent
Device rigidity or flexibility, dimensions, length, biocompatibility
Optimization of device insertion, imaging, incorporation of other functions
Drug injection device design, speed, and force of drug injection, which disperses drug optimally in tissue
Balloon design, which optimizes light delivery and reduces dislodgment risk
Light delivery optimization and determination of effective fluence rate, and light delivery methodology

The above list is not all inclusive of all of the development steps towards device and drug function optimization and is not intended to restrict other tests, tasks, and steps that may be required as well.

A physical or virtual tissue phantom is also created as an aid for treatment planning Device trajectory and entry into the lesion, drug delivery, and light delivery are all simulated using the physical model or image and planning software. Other modeling techniques and methods are known as well, which utilize various algorithms, calculations, and other means of simulations that approximate realistic tissue conditions and composition, drug and light dosing, and expected biological effects. Very accurate three- dimensional renditions of mass lesions can be created using current imaging techniques and imaging software. The optical properties of various tissues including tumorous tissues have been determined, and can be used to simulate light distribution and expected treatment effects at various drug dosages.

Embodiments described herein are multifunctional, which enables and facilitates proper device placement, drug delivery, imaging of effect, and drug activation with monitoring, which is novel.

The single pass into a tissue neoplasm, which may be malignant or benign, is followed by effective treatment of a large volume of tissue, reducing the risks of multiple device passes into tissue, tumor track seeding, bleeding, infection, pain and discomfort. This teaches away from the use of multiple, closely spaced, light fiber placements.

A single device can be used to treat a large tissue volume or region. Again, this embodiment teaches away from multiple, closely spaced, light fiber placements. Drug enhancements, which promote drug dispersion together with optical clearance are not described regarding treatment of large internal tumors. Follow up drugs such as immune enhancers can be administered if desired after the drug activation process using the same device. The use of tissue phantoms and simulations described herein can reduce the risk of harm and increase efficacy of treatment.

Prior PDT use related to the treatment of large internal masses teaches that a very prolonged illumination time along with multiple, closely spaced light sources are required for large volume disease. Systemic drug therapy is also taught by prior PDT art for sizeable mass lesion treatment. Very high total doses of light, delivered rapidly utilizing multiple light sources, is also previously taught in order to reduce treatment time. Clinical efficacy is extremely limited however, and the technique is impractical and not utilized. The present disclosure teaches away from past methods by using a very low drug dose delivered locally, which is orders of magnitude less than the typical systemic doses. The total light dose is low as well, and is delivered in a relatively short time span, which teaches away from prior use where only very small lesions are treatable in this manner. The use of a single device, even for a large lesion, which is enabled by the present disclosure, is a significant innovation.

The device can be positioned at the treatment site by way of percutaneous insertion, through a natural or artificially created orifice, stoma, or channel, or through the vascular system (arterial or venous). The positioning can be performed manually, robotically, endoscopically, using all types of image guidance, freehand placement, or in an automated or programmed fashion.

When utilizing intravascular access, the device can be inserted and guided using standard guidewires and image directed techniques. Once at the treatment site, PDT/LADT can be performed on the proximal aspect of the lesion, followed by advancement of the device into the parenchyma of the lesion in order to treat the remainder. Transitioning from the intravascular location to the interstitial, intratumoral region is enabled due to closure of vessels due to prior light activation, which obviates bleeding at the vascular-parenchyma transition point.

When utilizing endoscopic target lesion access, it is most convenient to remove the endoscope after device insertion. Otherwise, sedation or anesthesia required when using endoscopes of various types and in various locations must be maintained to prevent patient movement, maintain patient comfort, and to prevent device dislodgement. The proximal end of the light source can be discontinuous from the power source or laser module/laser diode assembly, enabling the endoscope to be backed away and uncoupled from the light source as it is withdrawn. After the endoscope is removed, the light source is reconnected to the proximal portion of the device to enable light activation.

The device can be composed of known MRI and CT compatible materials, which reduce the possibility of unwanted device movement and imaging artifact. Miniaturized MRI catheter coils or ultrasound transducers can also be optionally incorporated into the distal end of the device.

Any of the components of the device, which are indwelling in the body, can be constructed with biocompatible and biodegradable materials, and left behind after the termination of the treatment, if desired. For example, the sheath or catheter, the optical fiber, and the balloon can be detachable, or otherwise separable. Biodegradable polymers and substances are well known, as are detachable balloons. Leaving device components behind such as the sheath allows for ease of re-access to the treatment site in the future if required.

### Summary of Characteristics

The present disclosure reveals the utilization of a single entry point on the body and a single intracorporeal access route with localized drug delivery and light delivery combined to a large lesion volume (>2 cm). The present disclosure teaches away from the need for multiple access points and routes previously required to treat large lesions. A variety of embodiments, methods, and novel drug formulations enable treatment of large lesions using a single device, by way of increasing drug delivery and dispersion and light penetration in a larger than expected volume of tissue.

In an embodiment, the risk of harm may be reduced by having localized drug and light delivery combined, in contrast to systemic drug delivery with localized light delivery. An extremely high drug concentration is enabled by local injection if desired (much greater compared to systemic drug injection), which then allows for a short illumination time. This is due to the reciprocity principle. Novel drug formulations using optical clearing agents and drug dispersants and penetration enhancers such as DMSO are enabled in the present disclosure. The short illumination period enhances the ability to perform multiple light activation procedures within the large lesion within a reasonable clinical time frame, and without undue and impractical demands on imaging facilities and equipment.

### EXAMPLE 1

A subject (animal or human) in need of treatment of a mass lesion (malignant or benign) undergoes testing and imaging of the lesion in order to determine the safest, easiest route of access. Access in all examples can be percutaneous, intravascular, via a natural or created orifice or tunnel.

### EXAMPLE 1A

A needle capable of delivering drug through perforations in the distal needle shaft is percutaneously placed into the central portion of the lesion utilizing standard image guidance, either using CT, MRI, or ultrasound. Other means of localizing the device using navigation and localization instruments and techniques may be used as previously described as well. The needle is the conduit for PD, which is injected at a predetermined rate, and in a predetermined volume by a syringe reversibly connected to the proximal end of the needle. The drug injection process disperses the drug within the lesion. The needle is then used to position an optical fiber with a cylindrical diffusing tip, which is connected to a laser diode or laser. The optical fiber is inserted into the needle, positioned and held in place while the needle is withdrawn, exposing the cylindrical diffuser tip. The drug has continued to disperse within the lesion during this process, and is now activated by the laser light emanating from the diffuser tip. The lesion is treated by a predetermined light dose, and retreatment performed as needed, which is determined by the response. Alternatively, laser light is aimed towards the lesion interior from the edge of the lesion. In this instance, the optical fiber is positioned at the periphery of the lesion after withdrawing the needle device.

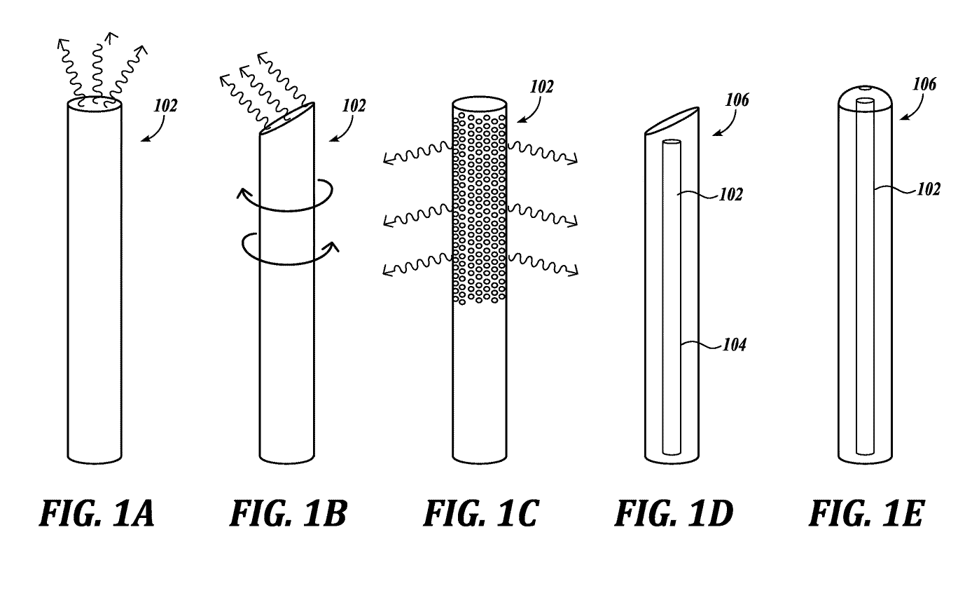

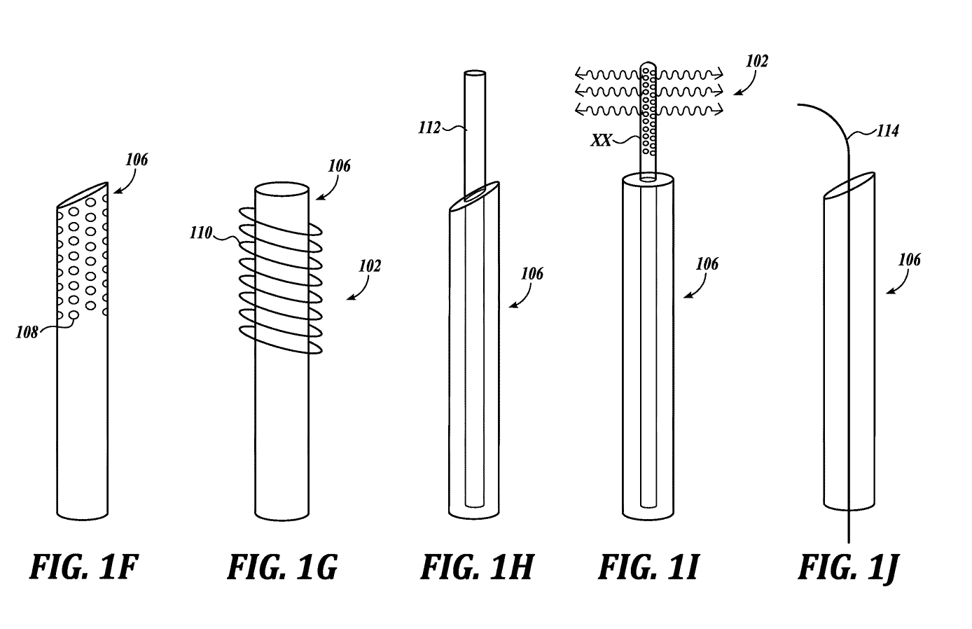

FIGS. 1A through 1J show portions of medical device in which one or more methodologies or technologies can be implemented, for example, treating abnormal tissues including cancer utilizing light activated drug therapy.

FIG. 1A shows a portion of medical device having a photo-activation assembly including a clad optical fiber 102 configured for forward aim.

In an embodiment, the photo-activation assembly includes circuitry configured to generate one or more parameters for incrementally increasing a light dose. In an embodiment, the photo-activation assembly includes circuitry configured to generate one or more parameters for delivering a light dose sufficient to induce programmed cell death, without substantially inducing necrosis, of cells within one or more target regions proximate an outer surface of the needle. In an embodiment, the photo-activation assembly includes at least one optical emitter. Non-limiting examples of optical energy emitters include arc flashlamps, cavity resonators, diode-pumped solid state lasers, exciplex lasers, incandescent emitters, laser diodes, lasers, light-emitting diodes (e.g., organic light-emitting diodes, polymer light-emitting diodes, polymer phosphorescent light-emitting diodes, microcavity light-emitting diodes, high-efficiency UV light-emitting diodes, or the like), light-emitting transducers, optical energy emitters, photon emitters, quantum dots, radiation emitters, semiconductor lasers, and the like.

In an embodiment, the optical fiber 102 is advanced or retracted to enhance effect zone. FIG. 1B shows a portion of medical device having a photo-activation assembly including a clad optical fiber 102 configured for angled aim. In an embodiment, the optical fiber 102 is rotate either direction about an axis to enhance effect zone. FIG. 1C shows a portion of medical device having a photo-activation assembly including a cylindrical diffusing optical fiber 102. In an embodiment, the optical fiber 102 is advanced or retracted to enhance effect zone. FIG. 1D s shows a portion of medical device having a photo-activation assembly including an optical fiber 102 received within passageway 104 of a needle 106 having a beveled tip. FIG. 1E shows a portion of medical device having a photo-activation assembly including an optical fiber 102 received within passageway of a needle 106 having a blunt tip. FIG. 1F shows a portion of a medical device including needle 106 having a plurality of perforation 108 for delivering one or more of a photodynamic therapy composition, a photosensitizer composition, tissue penetration composition, an optical clearing agent composition, and the like. FIG. 1G shows a portion of a medical device including needle 106 having a wrap-around coil 110 for localization, navigation, orienting, and the like. FIG. 1H shows a portion of a medical device including an acoustic assembly 112 receivable within the passageway of a needle 106. FIG. 1I shows a portion of a medical device including needle 106 retracting to expose a cylindrical diffusing fiber 102. FIG. 1J shows a portion of a medical device including a pre-curved guide wire 114 received within a passageway, and exiting from a needle 106 in an off-axis direction.

Referring to FIGS. 1A through 1J, optical fibers with different designs and capabilities are utilized. Optical fibers can be variably flexible or stiff, with various lubricious coatings in order to facilitate passage into the treatment area and subsequent removal. Multiple fiber designs and configurations are well known, and enable light to be emitted in different directions, in different beam widths, or in a cylindrical fashion. A Referring to FIGS. 1A through 1J, different needle designs can be use according to one or more embodiments. It is understood that many designs and configurations are possible, which enable drug to be delivered in various directions, in various volumes, and in various distribution patterns. The size, shape, dimensions, and pattern of needle holes and perforations plays a major role in determining drug distribution, and can be optimized in vitro, using tissue phantoms, in vivo, and can be followed in realtime using imaging. Referring to FIGS. 1G through 1J, various means for localizing the needle, and directing needle, and other actions of the needle are depicted. Coil localization and ultrasound localization are just some of the means of determining device localization, and the drawings are not intended to limit device detection means. Referring to FIG. 1J, in an embodiment, a curved guidewire allows for access to a lesion which is off of the axis of the needle.

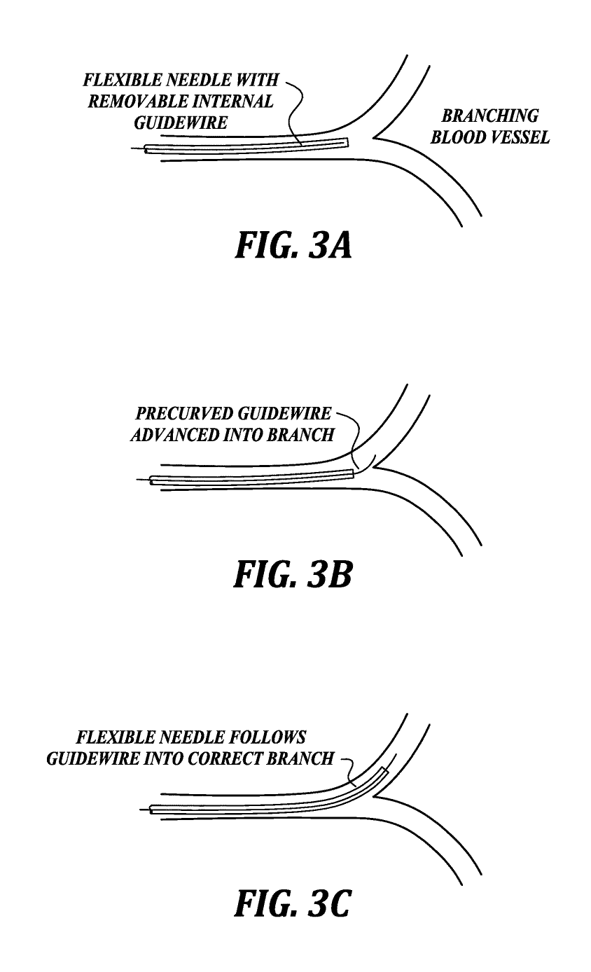

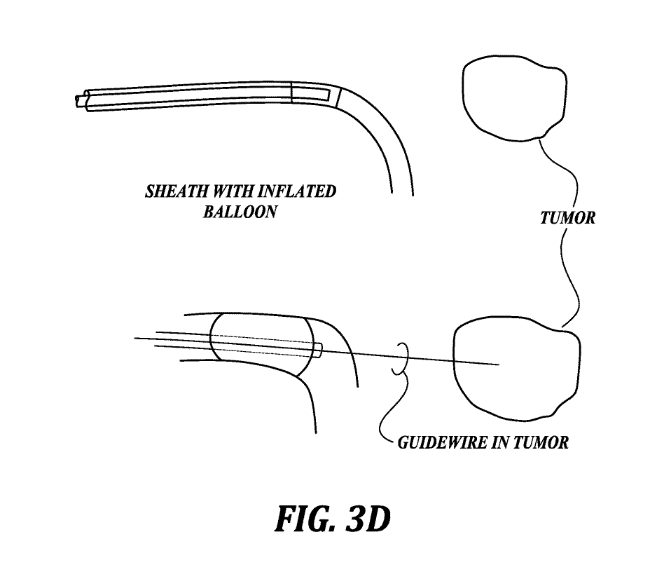

Referring to FIGS. 3A through 3D, in an embodiment, the use of flexible needles, guidewires, and sheaths/catheters are illustrated for intravascular use. The use of precurved, or other steerable guidewire and needle designs enables vascular or other tubular organ lumens to be utilized as access routes, which enables more reliable device fixation, reducing device migration risk.

### EXAMPLE 1B

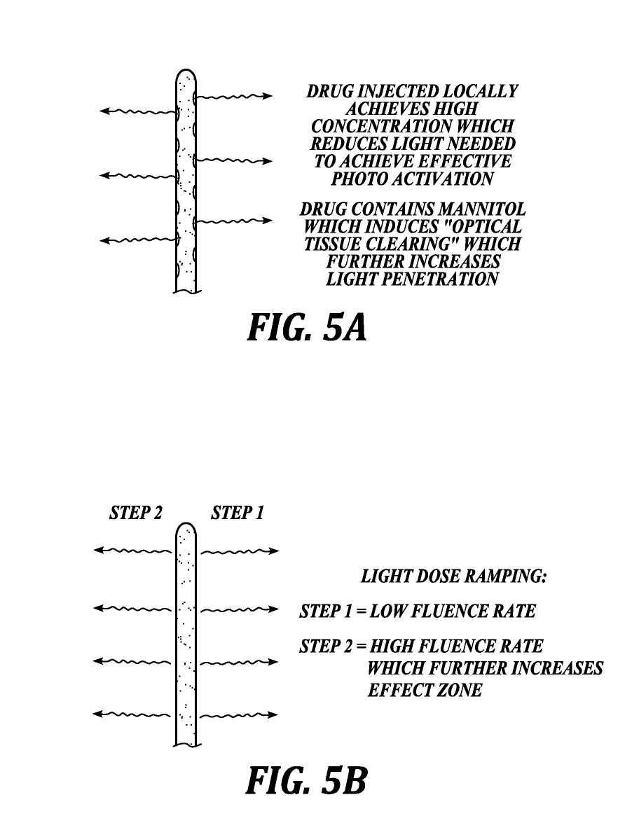

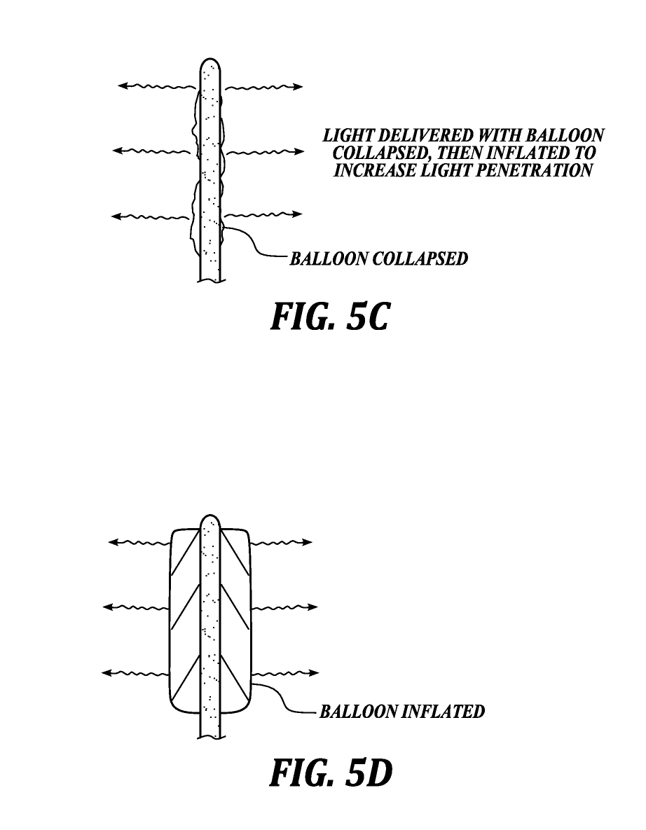

Referring to FIGS. 5A through 5C, in an embodiment, PD, which can be comprised of any of the PDs, is diluted with a mannitol solution, or other substances with optical clearing properties previously mentioned prior to injection. The mannitol serves to increase light penetration within the lesion, which in turn enlarges the treatment zone, beyond that enabled by the drug alone in a standard diluent, for example, saline. An additional benefit is that the enhanced light penetration reduces the illumination time, which reduces the overall procedural risk and increases convenience ad clinical utility and application. In addition to mannitol, dimethyl sulfoxide (DMSO) can used as a diluent for the photodynamic drug. DMSO has the advantage of enhancing drug dispersion in tissue, and at the same time increasing optical penetration of light. In an embodiment, high drug concentrations are achieved with local injections, which in turn, reduce the necessary illumination time, reducing risk, and improving clinical practicality.

### EXAMPLE 1C

Light is delivered a stepwise fashion from the optical fiber starting at a reduced fluence rate for a specified time. The photoactivation effect at the reduced fluence rate results in effective blood vessel closure and avoids too rapid oxygen consumption, which occurs at high fluence rates. The fluence rate is increased for another period of time followed by setting the light source at the highest fluence rate for the final light delivery interval. Referring to FIG. 5A through 5C, in an embodiment, overtreatment of the zone nearest the light source is avoided, and the intermediate and outermost treatment zone receives adequate light, even allowing for the expected light attenuation at a distance from the light source. Further, the light dose rate and amount can be altered during the therapy in cases where the treatment is performed using local anesthesia or very light patient sedation. Altering the light dose as treatment is proceeding can reduce patient discomfort, and is not specifically taught in prior PDT art.

### EXAMPLE 1D

A pre-curved, sharp, tissue penetrating wire is inserted into the rigid needle sheath, which has previously been placed at the treatment site. The wire, which essentially serves as a guide wire is advanced into the lesion and is angled or curved off of the main axis of the rigid needle. The rigid needle is withdrawn and replaced by a flexible needle, used to inject drug, and position an optical fiber or other light source off of the original rigid needle axis. In an embodiment, technologies and methodologies enable enlargement of the treatment zone using a single percutaneous entry point.

### EXAMPLE 1E

A rigid, sharp guidewire is inserted into a flexible needle sheath and introduced percutaneously into tissue, using image guidance. The total stiffness of the assembly allows penetration to the treatment site. Drug is injected using the needle sheath after removal of the guidewire. An optical fiber coupled to a laser diode is inserted into the needle sheath and the sheath is retracted to expose the optical fiber tip. The treatment site is illuminated. Then the optical fiber is withdrawn and replaced by a precurved, flexible guidewire, which is inserted into the needle sheath, and extended past the distal end of the needle sheath in the desired new direction. The flexible needle sheath is pushed forward and now follows the new path. Another drug dose can be injected, and the optical fiber can be repositioned in the new location for further photoactivation.

### EXAMPLE 1F

After the optical fiber or distal light emitting diode array is positioned at the treatment site, a balloon is inflated which fixes the device in place and also increases the effect zone and size. The balloon is deflated after illumination, facilitating device withdrawal. The inflated balloon also enables enhanced visualization of device location due to the altered signal, density, or echogenicity of the balloon within tissue. This reduces the chance for inadvertent illumination in the wrong location.

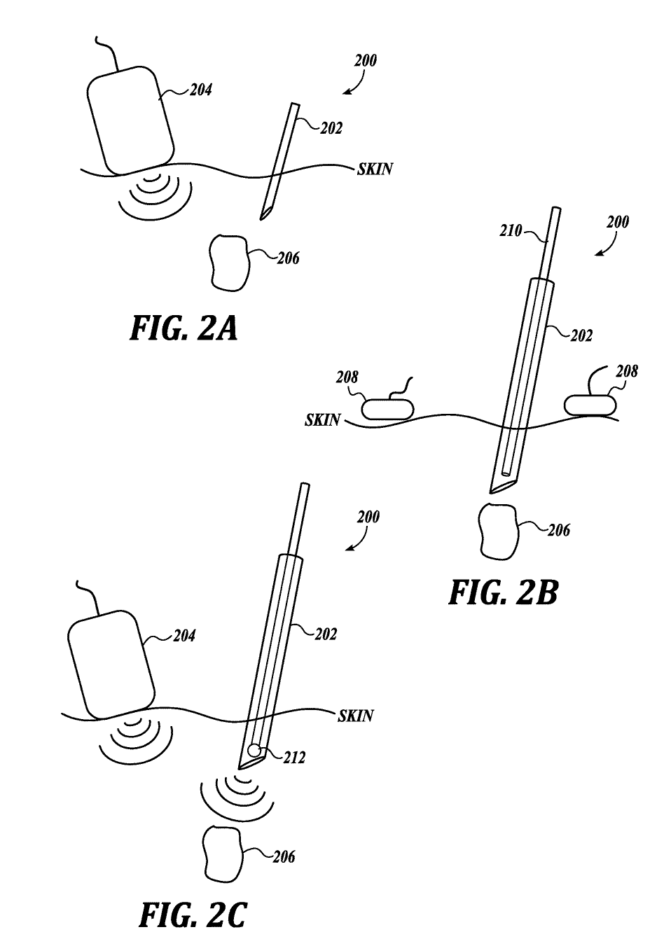

FIGS. 2A through 2C show portions of medical device in which one or more methodologies or technologies can be implemented, for example, for localizing the medical device internally to treat abnormal tissues including cancer utilizing light activated drug therapy. FIG. 2A shows a portion of medical device 200 using an external image guided methodology employing and ultrasound device 204 for placing the needle portion 202 of the medical device within a target region. In an embodiment, the image guided methodology facilitates placing the needle portion of the medical device within a target region proximate or within a tumor 208. FIG. 2B shows a portion of medical device 200 using an external image guided methodology employing detectors 208 for placing the needle portion 202 of the medical device within a target region proximate or within a tumor 208. The guided methodology includes a needle 202 having a removable electromagnetic coil 210, sensor, and the like with detectors 208 located externally for placing the distal portion of the needle 202 within a target region. FIG. 2C shows a portion of medical device 200 using an external image guided methodology for placing the needle portion 202 of the medical device within a target region proximate a tumor. The guided methodology includes a needle 202 having an acoustic assembly 212 received within the passageway of the needle. In an embodiment, the acoustically guided methodology facilitates placing the needle portion of the medical device within a target region.

### EXAMPLE 2

In a subject with an abnormal growth or growths in need of treatment, the blood vessels feeding the lesion(s) are identified and mapped, imaging techniques such as, for example, MRI, CT, angiography, ultrasound, and the like. Referring to FIGS. 2A through 2C, in an embodiment, methodologies and technologies include percutaneous image guided approaches. A standard or nonstandard vascular access route is used for guidewire placement into a tumor feeding vessel. In an embodiment, the flexible drug delivery and light delivery catheter is delivered into the feeding vessel using the guidewire. In an embodiment, the flexible drug delivery and light delivery catheter is delivered into the feeding vessel using the Seldinger technique.

### EXAMPLE 2A

The device is place within the lesion, and after confirmation of correct device location, the light emitting source is introduced into the device and drug is slowly injected by way of the intra-arterial route. In this example the light source emits light in a forward direction, which treats the more distal portion of the lesion. As drug is injected, the device is gradually withdrawn during light activation, which in turn treats the more proximal portion of the lesion.

### EXAMPLE 2B

After drug is injected, the light is delivered to the region of tumor closest to the entry point of the tumor feeding vessel. After blood flow stasis has occurred, the device is advanced into the parenchyma of the lesion, followed by drug injection and light activation. At the transition zone from blood vessel to solid tissue, bleeding is prevented as the device is advanced due to blood vessel closure in this area resulting from the initial photoactivation process.

### EXAMPLE 3

A subject with one or more lesions accessible using an endoscope is treated by inserting the device into a working channel, which is part of the endoscope. The lesion is visualized, and the device with a sharp tip is used to puncture the lesion. The drug delivery device is positioned within the lesion interior, and drug is delivered within the lesion using the injection apparatus, which can be a syringe. As drug distributes within the lesion, the light source is positioned interstitially, or aiming directly at the lesion, from the lesion edge towards the interior of the lesion. Light activation occurs, and the lesion is adequately treated.

In all examples, it is understood that preclinical testing can be performed at a prior time, in order to optimize device design and performance. Intraprocedural monitoring and clinical outcomes can be used to optimize the treatment parameters based on clinical feedback. A wide variety of PD can be utilized in the current disclosure, and all exhibit varying photoactivation characteristics and biological effects, which can be tested in the preclinical and laboratory setting prior to clinical use. Preclinical testing using established lesion models are used to determine optimal drug and light dosing. Clinical testing may be required to optimize device design, drug formulation, application, and methodologies as well. It is also understood that more than one treatment may be required for a given lesion.

### EXAMPLE 4

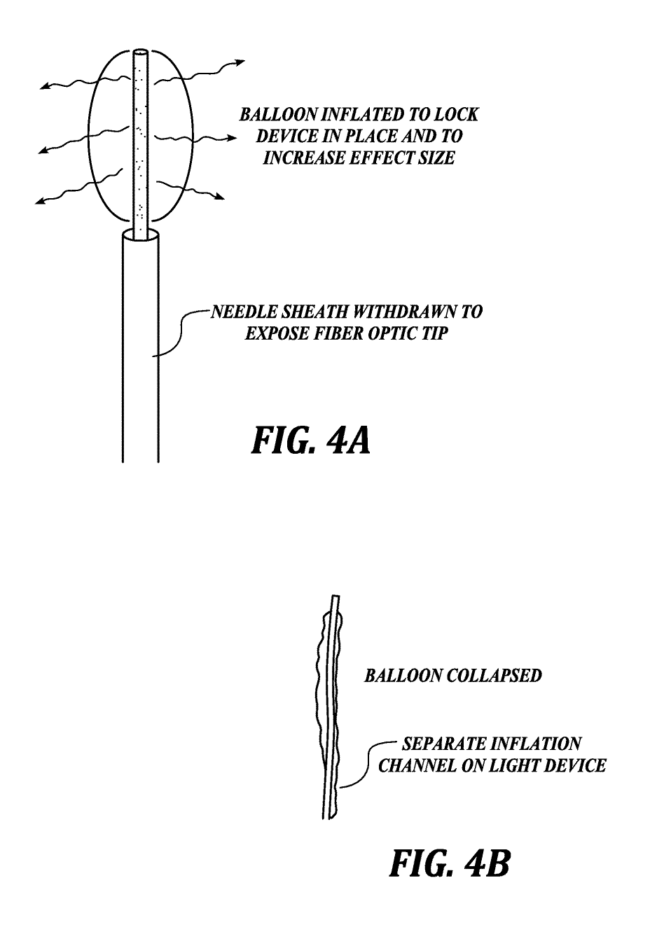

After the device assembly is correctly positioned in the target tissue using image guidance, the drug is locally delivered and light delivered by way of the unsheathed tip. Then a balloon, which is incorporated into the distal tip of the light device is inflated, which acts to increase the light zone of effect, and simultaneously prevents overtreatment of the tissue closest to the light source, as shown in FIGS. 5A through 5D. Inadvertent dislodgment of the light device is prevented, and after therapy is complete, the balloon is deflated and the device is explanted. In all cases of balloon use, the size, shape, dimensions, wall thickness of the balloon, and inflation pressures are determined and optimized in terms of desired light delivery capacity and capability using preclinical and lab experiments. Non-limiting examples of suitable fluids to inflate the balloon include air, light transmissible liquids, biocompatible liquids, and the like. FIGS. 4A and 4B also depict this embodiment.

### EXAMPLE 5

In an embodiment, the various guidewires, sheaths, needles, connectors, other suitable device components and optical fibers can be manufactured using a 3-D printer. In an embodiment, a wide variety of metallic and polymeric shapes are manufactures using 3-D printing. In an embodiment, using these printing devices, customized therapy systems can be made locally or on site, for example at the hospital where the patient resides, which affords convenience and economic advantages. Wires, needles, sheaths, optical components such as fibers, coils, and other parts described in this disclosure can be manufactured on demand, and in different lengths, thicknesses, and other dimensions. Stiffness and rigidity of various components can also be varied to accommodate insertion in to hard tissues such as bone, or soft tissues such as liver. Customization of device components is enabled, and can be produced in virtually any quantity on demand, which obviates need to maintain significant inventory, and the need for limited, costly production runs.

### EXAMPLE 6

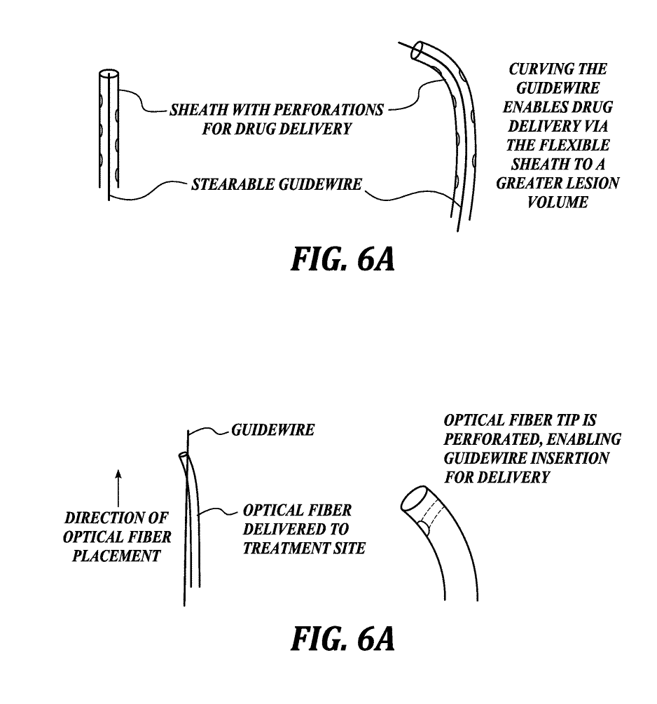

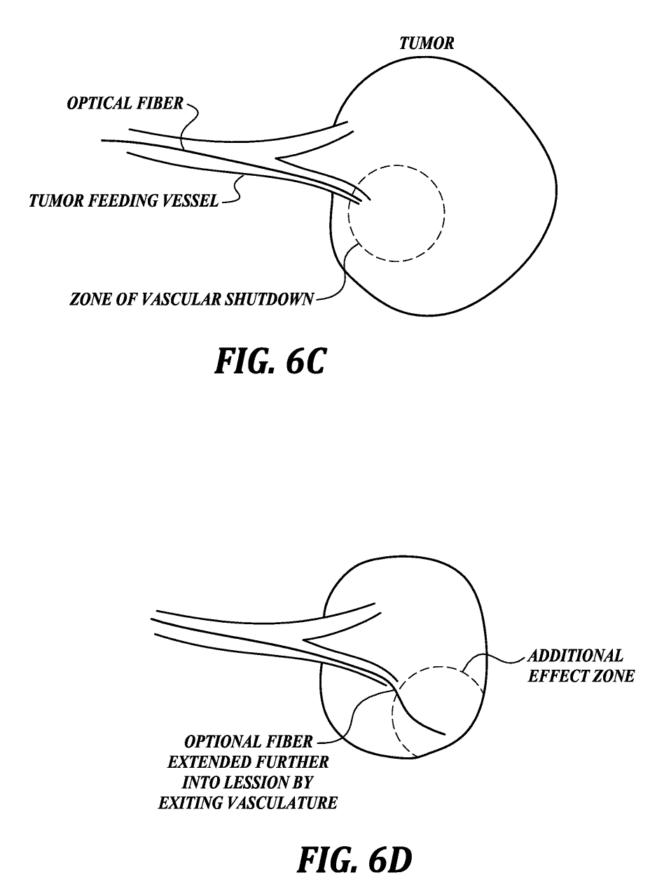

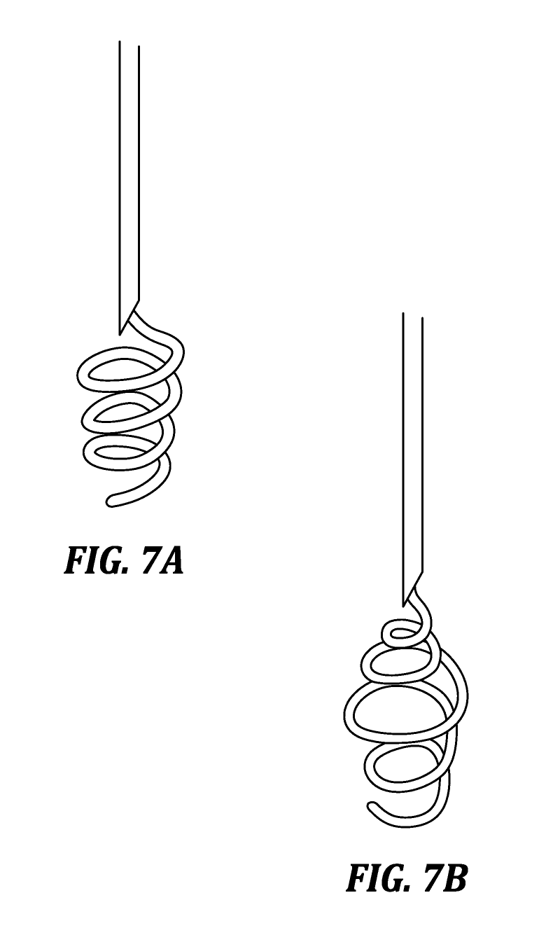

A rigid needle tipped (stiff guidewire) is used to percutaneously access the treatment site, which is a very large, bulky lesion. Referring to FIGS. 6A and 6B, a flexible sheath is inserted over the guidewire, or alongside the guidewire (monorail type also shown in FIGS. 6A and 6B) and PD is injected. The optical fiber coupled to a laser diode is introduced and positioned within the lesion. Illumination is performed, and then the optical fiber is backed out. Then a flexible, steerable wire is introduced through the flexible sheath, and angled such that a different, untreated portion of the lesion is accessed, which is off angle when referenced to the original axis and direction of treatment, as shown in FIGS. 6C and 6D). The flexible sheath is inserted, following the new path, and more PD is injected. The optical fiber is repositioned, and the new treatment zone is illuminated. Thus, using a single percutaneous access point, a very large volume of abnormal tissue is treated, and a minimal amount of tissue is traversed. By curving the guidewire, or retracting and redirecting the guidewire, more of the lesion volume is treated, and risks, including tumor tracking and seeding are reduced. This novel technique and apparatus also minimizes the risk of bleeding, reduces pain and discomfort, and allows for the safest access point on, and optimal trajectory, within the body.

### EXAMPLE 7

Using intravascular access, a flexible, steerable guide wire is utilized to gain access to the previously identified tumor feeding vessel, at the tumor border. Either the femoral artery or radial artery, or other arterial route can be utilized. The sheath with an optical fiber/laser is positioned, and drug is slowly injected through the tumor feeding artery. As this is performed, an optical fiber with a forward facing direction of illumination is used to illuminate the adjacent portion of the lesion. After this treatment, the tumor vessels are closed, which allows the guidewire to be advanced into the substance of the lesion, in an extravascular route and direction. Bleeding is obviated by prior vessel closure. The sheath is used to deliver PD interstitially at this point, and a different optical fiber with a cylindrical diffusing capability introduced and used to illuminate the remainder of the lesion, which is deep and distal to the first treatment zone. In this way, the targeted lesion is effectively treated using a combined intra and extravascular route, which minimizes the number of percutaneous punctures, and minimizes the thickness of solid tissue which must be penetrated. This maximizes avoidance of critical anatomic structures, which reduces the risk of harm of the procedure. FIGS. 3A through 3D depict various guidewires and catheter and needle placements in the intravascular compartment, which can be used to access previously identified tumor feeding vessels.

FIGS. 6C and 6D illustrate the use of tumor feeding vessel as the access route for the device. Tumor feeding vessels are identified using contrast dye angiograms, CT or MRI angiograms. The treatment effect zone is extended by inserting the guidewire more deeply into the lesion, under image guidance, after initial treatment at the periphery of the lesion induces vascular closure, also shown in FIGS. 6C and 6D.

### EXAMPLE 8

In patients with leukemia, the device can be utilized to access the bone marrow compartment, especially of the large long bones and pelvic bones. A large bore hollow needle is used to percutaneously access a convenient bone access site. A trocar or drill inserted within the hollow needle can be used to penetrate the cortex of the bone, and the drug and light delivery device inserted percutaneously. Positioning the device within the shaft of the bone allows for local PD delivery, followed by illumination within the marrow compartment. All of the major bones can be accessed in this manner, and the marrow can be treated with the aim of killing the cancerous cells within, and enabling growth of systemically infused normal stem cells in place of the leukemic cells. Normal stem cells, previously harvested and expanded in numbers, could also be reinfused directly into the previously treated bone marrow compartment, now devoid of the abnormal leukemic cells, which otherwise would crowd out the normal stem cells. Using this methodology, varying degrees of normal blood cell counts and function can be maintained. A similar technique can be used to treat solid tumors of the bone as well.

### EXAMPLE 9

A patient with a large plexiform neurofibroma and several large cutaneous neurofibromas which are inoperable is need of treatment. Using US guidance, the device is inserted into the substance of each lesion. Major nerve trunks are identified using pre-procedural imaging and avoided during percutaneous device placement. The drug is injected, followed by light activation. During the procedure, US monitoring is utilized to ascertain the magnitude of the treatment effect and nerve damage is avoided. The intensity of treatment is modulated by altering the light dose parameters during treatment, so as to minimize patient discomfort. The lesions are effectively treated, and retreatment of residual or new lesions is feasible at any future point.

### EXAMPLE 10

A patient with a large brain tumor located in a nonoperable location, with significant peritumoral edema, is need of therapy. The device is inserted through a burr hole into the tumor using image guidance and PD is injected. The subsequent photoactivation process closes the abnormally leaky tumor blood vessels, which reduces edema and improves neurologic function of the patient. The tumor is reduced in size over a period of time, and repeat treatments are rendered as necessary. The patient is treated with concomitant immunotherapy, which further destroys more tumor mass, especially at the tumor periphery. The device allows for real-time monitoring of the zone of effect, which serves to avoid damage to surrounding normal and critical brain tissue.

### EXAMPLE 11

A patient with widely metastatic cancer especially to bone is need of therapy. The device is used under image guidance in order to access the diseased cancerous bone, and therapy can be administered to the interior of the bone, with the device being advanced over a period of time, within the bone, in order to treat a large volume of tumor over a long distance. In a long bone, the device is advanced within the marrow compartment. In the spine, the device is advanced within the vertebral body, and can be guided to multiple vertebral levels from within the vertebrae, advancing in a cephalad or caudad direction, parallel to the long axis of the spine. This is accomplished utilizing an orthopedic drill, a long needle, a long catheter, and a long light source such as an optical fiber, whose length accommodates the length of the lesion and distance to placement. In this manner, multiple vertebral segments can be effectively treated, along with affected long bones, the pelvic bones, or even the ribs and the skull.

### EXAMPLE 12

A patient with leptomeningeal cancer spread is in need of treatment. The device is inserted into a cerebrospinal fluid (CSF) space, using an established route such as the frontal horn of the lateral ventricle as in a shunt placement, or by way of the lumbar CSF space as in a lumbar puncture. Multiple device can be placed within the CSF space if needed for increased illumination of diseased tissue surfaces. The PD drug is administered into the CSF, and the device provides illumination within the CSF space, which transmits light in all directions within the CSF compartment, including to remote CSF spaces such as the intradural spinal space and the cerebral ventricles. The transmitted light combined with the PD kills tumor cells within the leptomeninges without injuring sensitive, vital normal neurologic tissue. Overtreatment of brain or spinal cord is avoided using intra-procedural monitoring and/or serial neurologic examinations.

### EXAMPLE 13

A patient with a large internal tumor is considered for treatment using a conventional percutaneous approach. No safe percutaneous access route is found to be available, so the patient undergoes vascular imaging in order to determine the availability of a tumor feeding vessel. In this case, no obvious accessible tumor feeding vessels are imaged, so the patient undergoes intravascular device access with device exit at the vascular segment with the smallest lumen, into the extravascular space in close proximity to the tumor. Inflation of the balloon component of the device in the vessel lumen prevents bleeding, and the elongate balloon shape also serves to guide the guidewire into the interior of the tumor as it is advanced. Image guidance is used in order to direct the guidewire and confirm its location. A separate sheath is advanced over the guidewire for PD delivery, followed by insertion of a suitable optical fiber for illumination. After illumination, prior to balloon deflation, the device is withdrawn except for the outer sheath containing the balloon. The portion of the vessel where the device exited is thrombosed using an injected occlusive or clotting agent or substance, which are known. Then the remainder of the device ensemble is withdrawn. FIG. 3D depicts a catheter or sheath with a balloon incorporated, which when inflated, enables intravascular to extravascular placement of a guidewire into a lesion without direct tumor feeding vessel access. The guidewire serves to direct another drug delivery catheter to the treatment site, and subsequent placement of an optical fiber or other light source for drug photoactivation. Prior to withdrawal of the initially placed catheter or sheath, a clotting or occlusive agent such as Gelfoam or a biocompatible acrylic substance is injected at the vessel puncture site to prevent bleeding when the entire device is withdrawn.

### EXAMPLE 14

In a patient with a mass lesion without an accessible tumor feeding artery, a vascular conduit close to the treatment site is accessed and the lesion is treated, using a catheter incorporating a detachable balloon. At the conclusion of treatment the balloon is detached, leaving the vessel occluded which prevents bleeding. The balloon is optionally composed of a bioresorbable material. The catheter is also optionally composed of a bioresorbable material and can be left in situ, especially if an alternative percutaneous route was originally used. In this instance the catheter is cut proximally such that access is enabled subcutaneously on a repeat basis as needed. In this case, the proximal end of the catheter can be connected to an access port, which is subcutaneously positioned, which enables both drug injection into the catheter, and access by a light source. The bioresorption rate can be adjusted to occur over weeks. If the lesion requires repeat treatment the proximal catheter is accessed to enable drug injection and reinsertion of an optical fiber or light source.

Repeat therapy using the same access route and intralesional location of the catheter/sheath and light source may be useful as the original lesion shrinks in size due to gradual involution. As this process occurs, the lesion's perimeter becomes accessible for further treatments.

In an embodiment, technologies and methodologies significantly expands the zone of PDT/LADT treatment effect enabled by a single device, utilizing a single entry point, thus teaching away from prior use which enables only a very limited or very superficial treatment effect. Previously used PDT/LADT devices do not enable the effect zone expansion as detailed in the present disclosure.

In an embodiment, technologies and methodologies teach away from the use of multiple light sources within a localized lesion, whose placement is very complex, and whose use adds risk and discomfort to the procedure. In contrast, the present disclosure teaches very limited or a single light source, which is simpler and decreases the risk of harm. In an embodiment, technologies and methodologies monitor the treatment in real time, along with methods of localizing the device internally, and reduce risk of device migration. Rather than using optical clearing for a thin surface such as skin, in an embodiment, technologies and methodologies improves on the drug tissue interaction by enabling increased light transmission within the drug infused tissue, which reduces the required illumination time, thus facilitating clinical utility.

In an embodiment, technologies and methodologies improve on drug dispersion from a single device by incorporating the use of a drug penetration enhancer, not taught previously, which instead teaches systemic drug delivery for treatment of larger lesions.

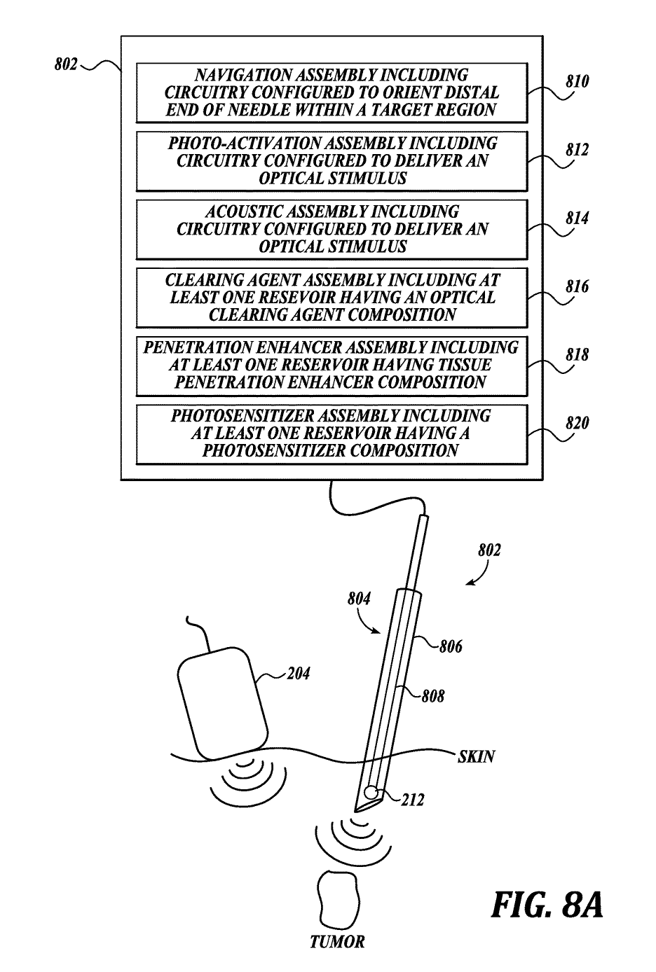

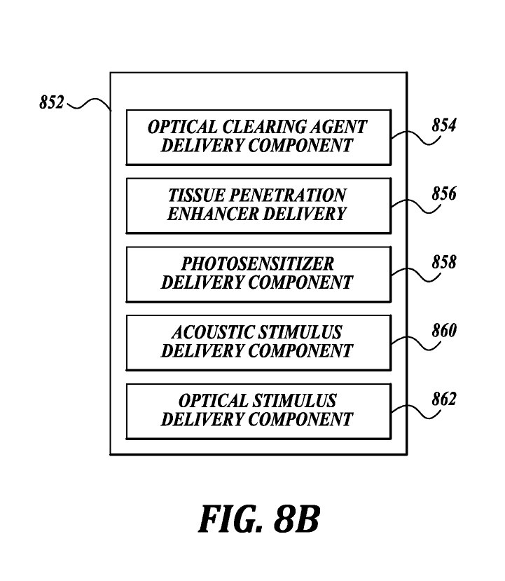

Referring to FIG. 8A, in an embodiment, a medical device 802 includes a needle 804 having body structure 806 having at least one internal surface defining a passageway 808. In an embodiment, the medical device includes a navigation assembly 810 including circuitry configured to orient a distal end of needle within a target region.

In an embodiment, circuitry includes, among other things, one or more computing devices such as a processor (e.g., a microprocessor), a central processing unit (CPU), a digital signal processor (DSP), an application-specific integrated circuit (ASIC), a field programmable gate array (FPGA), or the like, or any combinations thereof, and can include discrete digital or analog circuit elements or electronics, or combinations thereof.

In an embodiment, circuitry includes one or more ASICs having a plurality of predefined logic components. In an embodiment, circuitry includes one or more FPGA having a plurality of programmable logic components. In an embodiment, circuitry includes one or more sensors, detectors, transducers, and the like. Non-limiting examples of sensors include acoustic sensors, optical sensors, electromagnetic energy sensors, image sensors, photodiode arrays, charge-coupled devices (CCDs), complementary metal-oxide-semiconductor (CMOS) devices, transducers, optical recognition sensors, infrared sensors, radio frequency components sensors, thermo sensors, or the like. In an embodiment, the medical device 802 includes circuitry having one or more components operably coupled (e.g., communicatively, electromagnetically, magnetically, ultrasonically, optically, inductively, electrically, capacitively coupled, or the like) to each other. In an embodiment, circuitry includes one or more remotely located components. In an embodiment, remotely located components are operably coupled via wireless communication. In an embodiment, remotely located components are operably coupled via one or more receivers, transceivers, or transmitters, or the like.

In an embodiment, circuitry includes one or more memory devices that, for example, store instructions or data. For example, in an embodiment, the medical device 802 includes one or more memory devices that store one or more parameters for orienting and optical stimulus, for locating a needle within a biological subject, for delivering photodynamic therapy, and the like. Non-limiting examples of one or more memory devices include volatile memory (e.g., Random Access Memory (RAM), Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like), persistent memory, or the like. Further non-limiting examples of one or more memory devices include Erasable Programmable Read-Only Memory (EPROM), flash memory, or the like. The one or more memory devices can be coupled to, for example, one or more computing devices by one or more instructions, data, or power buses.

In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, circuitry includes one or more user input/output components that are operably coupled to at least one computing device to control (electrical, electromechanical, software-implemented, firmware-implemented, or other control, or combinations thereof) at least one parameter associated with, for example, delivering photodynamic therapy.

In an embodiment, circuitry includes a computer-readable media drive or memory slot that is configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as a magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., receiver, transceiver, or transmitter, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In an embodiment, the medical device includes a photo-activation assembly 812 receivable within the passageway. In an embodiment, the photo-activation assembly 812 includes at least one optical emitter. In an embodiment, the photo-activation assembly 812 includes circuitry configured to generate one or more parameters for incrementally increasing a light dose. In an embodiment, the photo-activation assembly 812 includes circuitry configured to generate one or more parameters for delivering a light dose sufficient to induce programmed cell death, without substantially inducing necrosis, of cells within one or more target regions proximate an outer surface of the needle.

In an embodiment, the medical device 802 includes an acoustic assembly 814 receivable within the passageway. In an embodiment, the acoustic assembly 814 includes at least one transducer operable to deliver an acoustic stimulus to one or more target regions proximate an outer surface of the needle. In an embodiment, the medical device includes a clearing agent assembly 816 receivable within the passageway. In an embodiment, the clearing agent assembly 816 includes at least one reservoir having an optical clearing agent composition. In an embodiment, the clearing agent assembly 816 is configured to deliver the optical clearing agent composition from the at least one reservoir to one or more target regions proximate an outer surface of the needle. In an embodiment, the optical clearing agent composition includes one or more materials that reduces light scattering of tissue. In an embodiment, the optical clearing agent composition includes one or more of butanediols, dextrose, dimethyl sulfoxide, fructose, glucose, glycols, hypaque, mannitol, trazograph, and verografin.

In an embodiment, the medical device includes a penetration enhancer assembly 818 receivable within the passageway. In an embodiment, the penetration enhancer assembly includes at least one reservoir having a tissue penetration enhancer composition. In an embodiment, the penetration enhancer assembly 818 is configured to deliver the tissue penetration enhancer composition from the at least one reservoir to one or more target regions proximate an outer surface of the needle. In an embodiment, the tissue penetration enhancer composition includes one or more of DMSO and ethanol.

In an embodiment, the medical device includes a photosensitizer assembly 820 receivable within the passageway. In an embodiment, the photosensitizer assembly 820 includes at least one reservoir having a photosensitizer composition. In an embodiment, the photosensitizer assembly 820 is configured to deliver the photosensitizer composition from the at least one reservoir to one or more target regions proximate an outer surface of the needle. In an embodiment, the photosensitizer composition includes one or more of methylene blue, porphyins, chlorins, phthalocyanines, tetrapyrroles, psoralens, flavins, tetracyclines, sulfa compounds, and photoactive prodrugs.

Referring to FIG. 8B, in an embodiment, photodynamic therapy device 852 including an optical clearing agent delivery component 854. In an embodiment, the photodynamic therapy device 852 includes a tissue penetration enhancer delivery component 856. In an embodiment, the photodynamic therapy device includes a photosensitizer delivery component 858. In an embodiment, the photodynamic therapy device 852 includes an acoustic stimulus delivery component 860. In an embodiment, the photodynamic therapy device 852 includes an optical stimulus delivery component 862.

In an embodiment, a method includes generating at one or more parameter for controlling at least one of a direction, intensity, duration, or peak emission wavelength of an illumination source responsive to acoustic information indicative of a tumor location, a tumor dimension, or tumor type. In an embodiment, the method includes interrogating one or more regions within a biological subject with an optical stimulus. In an embodiment, the optical stimulus is of a character and for a duration sufficient to activate one or more photosensitizers within one or more regions within a biological subject. In an embodiment, interrogating the one or more regions within a biological subject with the optical stimulus includes incrementally increasing a light dose. In an embodiment, interrogating the one or more regions within a biological subject with the optical stimulus includes interrogating the one or more regions with an optical stimulus having a dose sufficient to induce programmed cell death, without substantially inducing necrosis, of cells within the one or more regions within a biological subject.

In an embodiment, the method includes delivering a photodynamic therapy composition including at least one photosensitizer, at least one optical clearing agent, and a pharmaceutical acceptable carrier. In an embodiment, the method includes delivering a photodynamic therapy composition including at least one photosensitizer, at least one tissue penetration enhancer, and a pharmaceutical acceptable carrier. In an embodiment, the method includes delivering a photodynamic therapy composition including at least one photosensitizer, at least one tissue penetration enhancer, at least one optical clearing agent, and a pharmaceutical acceptable carrier.

In an embodiment, a photodynamic therapy composition includes at least one photosensitizer and a pharmaceutical acceptable carrier. In an embodiment, the at least one photosensitizer includes one or more of methylene blue, porphyins, chlorins, phthalocyanines, tetrapyrroles, psoralens, flavins, tetracyclines, sulfa compounds, and photoactive prodrugs. In an embodiment, the photodynamic therapy composition includes at least one optical clearing agent and a pharmaceutical acceptable carrier. In an embodiment, the at least one tissue penetration enhancer includes one or more of DMSO and ethanol. Other nonlimiting tissue penetrant examples include short and long chain alcohols, amides, esters, fatty acids, fatty acid esters, liposomes, which may optionally contain photosensitizers, phospholipids, pyrrolidones, surfactants, urea and derivatives, cyclodextrins, oxazolidinones, monoolein, vitamin E, terpenes, iminosulfuranes, and the like. In an embodiment, the at least one optical clearing agent includes one or more of butanediols, dextrose, dimethyl sulfoxide, fructose, glucose, glycols, hypaque, mannitol, trazograph, and verografin. In an embodiment, the photodynamic therapy composition includes at least one tissue penetration enhancer and a pharmaceutical acceptable carrier.

While particular aspects of the present subject matter described herein have been shown and described, it will be apparent to the reader that, based upon the teachings herein, changes and modifications can be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to, ” the term “having” should be interpreted as “having at least, ” the term “includes” should be interpreted as “includes but is not limited to, ” etc.). Further, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations, ” without other modifiers, typically means at least two recitations, or two or more recitations). Furthermore, in those instances where a convention analogous to “at least one of A, B, and C, etc.” is used, in general such a construction is intended in the sense of the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense of the convention (e.g., “ a system having at least one of A, B, or C” would include but not be limited to systems that have A alone, B alone, C alone, A and B together, A and C together, B and C together, and/or A, B, and C together, etc.). Typically a disjunctive word or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of

“A” or “B” or “A and B.”

With respect to the appended claims, the operations recited therein generally may be performed in any order. Also, although various operational flows are presented in a sequence(s), it should be understood that the various operations may be performed in orders other than those that are illustrated, or may be performed concurrently. Examples of such alternate orderings includes overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. A medical device, comprising:
a needle having body structure including at least one internal surface defining a passageway;
a photo-activation assembly receivable within the passageway, the photo-activation assembly including at least one optical emitter; and
an acoustic assembly receivable within the passageway, the acoustic assembly including at least one transducer operable to deliver an acoustic stimulus to one or more target regions proximate an outer surface of the needle.

2. The medical device of claim 1, wherein the photo-activation assembly includes circuitry configured to generate one or more parameters for incrementally increasing a light dose.

3. The medical device of claim 1, further comprising:
a clearing agent assembly receivable within the passageway, the clearing agent assembly including at least one reservoir having an optical clearing agent composition; the clearing agent assembly configured to deliver the optical clearing agent composition from the at least one reservoir to one or more target regions proximate an outer surface of the needle.

4. The medical device of claim 2, wherein the optical clearing agent composition comprises one or more materials that reduce light scattering of tissue.

5. The medical device of claim 2, wherein the optical clearing agent composition comprises one or more of butanediols, dextrose, dimethyl sulfoxide, fructose, glucose, glycols, hypaque, mannitol, trazograph, and verografin.

6. The medical device of claim 1, further comprising:
a penetration enhancer assembly receivable within the passageway, the penetration enhancer assembly including at least one reservoir having a tissue penetration enhancer composition; the penetration enhancer assembly configured to deliver the tissue penetration enhancer composition from the at least one reservoir to one or more target regions proximate an outer surface of the needle.

7. The medical device of claim 6, wherein the tissue penetration enhancer composition comprises one or more of DMSO, ethanol, short and long chain alcohols, amides, esters, fatty acids, fatty acid esters, liposomes, which may optionally contain photosensitizers, phospholipids, pyrrolidones, surfactants, urea and derivatives, cyclodextrins, oxazolidinones, monoolein, vitamin E, terpenes, and iminosulfuranes.

8. The medical device of claim 1, further comprising:
a photosensitizer assembly receivable within the passageway, the photosensitizer assembly including at least one reservoir having a photosensitizer composition; the photosensitizer assembly configured to deliver the photosensitizer composition from the at least one reservoir to one or more target regions proximate an outer surface of the needle.

9. The medical device of claim 8, wherein the photosensitizer composition comprises one or more of methylene blue, porphyins, chlorins, phthalocyanines, tetrapyrroles, psoralens, flavins, tetracyclines, sulfa compounds, and photoactive prodrugs.

10. The medical device of claim 1, further comprising:
a navigation assembly including circuitry configured to orient a distal end of needle within a target region.

11. A method, comprising:
generating at one or more parameter for controlling at least one of a direction, intensity, duration, or peak emission wavelength of an illumination source responsive to acoustic information indicative of a tumor location, a tumor dimension, or tumor type; and
interrogating one or more regions within a biological subject with an optical stimulus, the optical stimulus of a character and for a duration sufficient to activate one or more photosensitizers within one or more regions within a biological subject.

12. The method of claim 11, wherein interrogating the one or more regions within a biological subject with the optical stimulus includes interrogating the one or more regions with an optical stimulus having a dose sufficient to induce programmed cell death, without substantially inducing necrosis, of cells within the one or more regions within a biological subject.

13. The method of claim 11, wherein interrogating the one or more regions within a biological subject with the optical stimulus includes incrementally increasing a light dose.

14. The method of claim 11, further comprising:
delivering a photodynamic therapy composition including at least one photosensitizer, at least one optical clearing agent, and a pharmaceutical acceptable carrier.

15. The method of claim 11, further comprising:
delivering a photodynamic therapy composition including at least one photosensitizer, at least one tissue penetration enhancer, and a pharmaceutical acceptable carrier.

16. The method of claim 11, further comprising:
delivering a photodynamic therapy composition including at least one photosensitizer, at least one tissue penetration enhancer, at least one optical clearing agent, and a pharmaceutical acceptable carrier.

17. A photodynamic therapy composition, comprising:
at least one photosensitizer;
at least one tissue penetration enhancer;
at least one optical clearing agent; and
a pharmaceutical acceptable carrier.

18. The photodynamic therapy composition of claim 17, wherein the at least one tissue penetration enhancer includes one or more of DMSO, ethanol, short and long chain alcohols, amides, esters, fatty acids, fatty acid esters, liposomes, which may optionally contain photosensitizers, phospholipids, pyrrolidones, surfactants, urea and derivatives, cyclodextrins, oxazolidinones, monoolein, vitamin E, terpenes, and iminosulfuranes.

19. The photodynamic THERAPY composition of claim 17, wherein the at least one optical clearing agent includes one or more of butanediols, dextrose, dimethyl sulfoxide, fructose, glucose, glycols, hypaque, mannitol, trazograph, and verografin.

20. The photodynamic therapy composition of claim 17, wherein the at least one photosensitizer includes one or more of methylene blue, porphyins, chlorins, phthalocyanines, tetrapyrroles, psoralens, flavins, tetracyclines, sulfa compounds, and photoactive prodrugs.

21. A photodynamic therapy device, comprising:
an optical clearing agent delivery component;
a tissue penetration enhancer delivery component; and
a photosensitizer delivery component.

22. The photodynamic therapy device of claim 21, further comprising:
an acoustic stimulus delivery component.

23. The photodynamic therapy device of claim 21, further comprising:
an optical stimulus delivery component.
